(19) United States
(12) Patent Application Publication
(10) Pub. No.: US 2002/0094576 A1
(43) Pub. Date: Jul. 18, 2002

(54) Material and methods relating to a novel retrovirus

(76) Inventors: David J. Griffiths, London (GB); Robert A. Weiss, London (GB); Patrick Venables, London (GB)

Correspondence Address: DANN DORFMAN HERRELL & SKILLMAN, SUITE 720, 1601 MARKET STREET, PHILADELPHIA, PA, 19103-2307, US

(21) Appl. No.: 09/920,552

(22) Filed: Aug. 1, 2001

Related U.S. Application Data
(63) Continuation-in-part of application No. 09/280,329, filed on Mar. 29, 1999.
(60) Provisional application No. 60/115,268, filed on Jan. 8, 1999.

(30) Foreign Application Priority Data
Mar. 27, 1998 (GB) GB9806649.1

(51) Int. Cl. C12N 15/867; C12P 21/02; C12N 7/00; C12Q 1/70; C12P 21/06; C12P 19/34; A61K 39/12; A61K 39/21; C12N 7/01; C07K 2/00; C07K 4/00; C07K 5/00; C07K 7/00; C07K 14/00; C07K 16/00; C07K 17/00; A61K 38/00; C12N 15/00; C12N 15/09; C12N 15/63; C12N 15/70; C12N 15/74; C07K 1/00; C12N 15/86

(52) U.S. Cl. 435/456; 435/69.1; 435/235.1; 435/69.5; 435/320.1; 424/199.1; 435/5; 530/300; 530/350; 435/91.1; 424/186.1; 424/187.1

(57) ABSTRACT

The present invention relates to a novel retrovirus associated with autoimmune disease. The present invention provides nucleotide and amino acid sequences relating to GAG, PRO and POL proteins of the retrovirus as well as diagnostic techniques and antibodies for use in diagnosis. The retrovirus (HRV-5) according to the present invention has been detected in inflamed joints (RA, osteoarthritis (OA), reactive arthritis and psoriatic arthritis) but not normal synovium. Further, HRV-5 proviral DNA has been detected in blood from patients with RA and systemic lupus erythematosus (SLE).

The present application is a continuation-in-part of U.S. application Ser. No. 09/280,329 filed Mar. 29, 1999, which claims priority under 35 U.S.C. §119(e) to GB9806649.1 entitled Material and Methods Relating to a New Retrovirus” filed Mar. 27, 1998 and U.S. Provisional Application, No. 60/115,268 of the same title, filed Jan. 8, 1999. The disclosures of all of the above-identified applications are hereby incorporated by reference as though set forth in full herein.

### FIELD OF THE INVENTION

The present invention concerns materials and methods relating to a novel retrovirus associated with autoimmune disease, as well as diagnostic techniques and kits, to antibodies which bind said retrovirus and their use in diagnosis. Also included are methods of treatment of autoimmune disease and compositions for use in those methods.

### DESCRIPTION OF RELATED ART

A substantial body of indirect data supports the hypothesis that a retrovirus may be the etiological agent in a range of autoimmune diseases such as rheumatoid arthritis (RA), Sjogren's syndrome (SS) and systemic lupus erythematosus (SLE), but convincing direct evidence is still lacking.

The hypothesis that retroviral infection has a role to play in the pathogenesis of RA is given support by research demonstrating that human T-cell lymphotropic virus type I (HTLV-I) (the etiologic agent of adult T-cell leukemia (ATL) and tropical spastic paraparesis (TSP)) is associated with an arthropathy (usually chronic and oligoarticular) that has many features in common with RA (Nishioka. K. et al. The Lancet 1989; I: 441). Mice transgenic for the HTLV-I tax gene also develop a polyarthritis resembling RA (Iwakura. Y. et al. Science 1991; 253: 1026-1028). These mice express high levels of the viral transactivator protein Tax in the joints along with high levels of interleukin-1α messenger RNA. Other reports have demonstrated that human synovial cells which are transgenic for the Tax protein show enhanced proliferative capacity and GM-CSF production (Sakai. M. et al. J. Clin. Invest.; 1993; 92: 1957-1966; and Nakajima. T. et al. J. Clin. Invest.; 1993; 92: 186-193) and also increased expression of IL-6 (Mori. N. et al. J. Rheumatol.; 1995; 22: 2049-2054). An animal model of spontaneous inflammatory arthritis induced by a retrovirus is the caprine arthritis encephalitis virus-infected goat. This disease shares some features in common with RA in that CAEV-induced lesions contain large numbers of inflammatory cells including activated macrophages, macrophage-like type A synovial cells and type B synovial fibroblasts in addition to T cells (Wilkerson. M. et al. J. Rheumatol.; 1995; 22: 8-15). CAEV infects monocytes and macrophages and proviral DNA has been detected at multiple sites (Zink. M. et al. Am.

J. Pathol.; 1990; 136: 843-854) suggesting that viral expression is dependent on the maturation state of monocytes, with macrophages in lesions showing high levels of viral gene expression. The present inventors have recently described the cloning and sequencing of a 930 bp fragment (JC96) of a new human retroviral genome from particles purified from tissues and cultured cells or tissues (Griffiths. D. et al. J. Virol. 1997; 71: 2866-2872). This sequence corresponded to part of a novel pol gene containing overlapping reading frames encoding part of the protease (PR) and reverse transcriptase (RT) enzymes. This information forms part of previously filed European application 96912159.9 in the name of Griffiths et al.

The similarity of JC96 to rodent IAP genes suggested that the novel retrovirus may encode the human IAPs reported by Garry et al (Garry. R. F.; et al 1990. Science 250: 1127-1129). However, rodent IAPs are thought to be encoded by endogenous retroviruses with defective envelope genes (Kuff, E. L.; et al 1988 Adv. Cancer Res. 51: 183-276). In addition, extracellular virions were not detected in the cultures studied by Garry et al (Garry. R. F.; et al 1990. Science 250: 1127-1129).

The low abundance of JC96 in tissues, the high level of sequence similarity between different isolates, and the maintenance of open reading frames for two of its enzymes support the hypothesis that the new retrovirus is part of an exogenous retrovirus.

Further studies on the retrovirus have been prevented by the inability to amplify similar sequences from human cell lines and tissues, due to its extremely low abundance. RT-PCR and standard PCR with degenerate primers based on other conserved regions of retrovirus genomes has proved unsuccessful in allowing the present inventors to extend the sequence into gag or env.

### SUMMARY OF THE INVENTION

The present invention relates to the further characterization of the novel retrovirus previously reported in European patent application number 96912159.9., U.S. Provisional Application 60/115,268 and U.S. application Ser. No. 09/280,329.

Unlike the large numbers of endogenous retroviral sequences which have already been described (Nakagawa. K. et al. Arthritis and Rheumatism 1997; 40: 627-638; and Patience. C. et al. Trends in Genetics 1997; 13: 116-120), this new retroviral agent has all the characteristics of an exogenous (i.e., infectious) agent. To date there are only four known infectious human retroviruses, HIV 1 and 2, and HTLV-I and II (the “human” foamy virus has recently been found to be a zoonosis) (Weiss R. A.; Nature 1996; 380: 201). The novel retrovirus has therefore been designated human retrovirus-5 (HRV-5) to which the present invention relates.

In the initial studies of the present inventors, HRV-5 RNA was detected in 6/18 cell or tissue homogenates layered on sucrose density gradients both from patients with SS and patients with normal salivary glands. This method however, although extremely sensitive, is not suitable for epidemiological studies. The present inventors realized that it was desirable to detect proviral DNA which is a much more robust assay, albeit less sensitive. As the original sequence was cloned from a patient with SS, the present inventors tested 97 salivary gland biopsies from patients and controls. Surprisingly, they found only two positives; one being from a patient with RA who also has secondary SS (Rigby et al.; Arthritis and Rheumatism 1997: 40:2016). The present inventors therefore concluded that HRV-5 was unlikely to replicate in salivary glands and so other autoimmune and inflammatory diseases were screened for the presence of HRV-5 proviral DNA sequences. In the course of these new studies, other primer sets were evaluated and from these HRV-5 was found to selectively concentrate in synovial tissues, particularly in inflamed joints.

However, even with this knowledge, the further characterisation of HRV-5 has only been possible since the design of a particular set of primers (“best” primers) which were more sensitive for detecting HRV-5DNA in clinical tissues than other primers used. It has only been through the use of these primers that further nucleotide sequence has been obtained from positive DNA samples.

The inventors have now detected HRV-5 proviral DNA in inflamed joints (RA, osteoarthritis (OA), reactive arthritis and psoriatic arthritis) but not normal synovium. Further, HRV-5 proviral DNA has been detected in blood from patients with RA, systemic lupus erythematosus (SLE) and inflammatory bowel diseases. This may be because the virus is tropic for cell types abundant in these tissues such as macrophages or fibroblasts. It is possible that some types of arthritis may be an unusual reaction to a common infection. Table I shows further disease states in which the present inventors has detected HRV-5.

Therefore, at its most general, the present invention provides materials and methods relating to HRV-5 for use in treatment, diagnosis or therapy of autoimmune diseases and other inflammatory diseases such as arthritis, and SLE.

In a first aspect the present invention provides a double-stranded nucleic acid molecule (SEQ ID NOS: 1 and 4, 127) which comprises a novel nucleotide sequence which encodes a peptide as shown in FIG. 1 (SEQ ID NOS: 1,2,3,4), FIG. 3 (SEQ ID NO: 5), FIG. 6 (SEQ ID NOS: 8,9,10,11) FIG. 10 (SEQ ID NOS: 12, 13, 14), FIG. 12 (SEQ ID NOS: 13, 15, 16, 17, 18), FIG. 13 (SEQ ID NOS: 19, 20) or FIG. 21, (SEQ ID NO: 103) or variants, mutants or fragments thereof.

FIG. 1 shows gag and protease (pro) genes (encoding nucleocapsid, dUTPase and the N-terminal part of protease). FIG. 3 shows the pol gene (encoding RT/RNaseH and integrase). FIG. 6 shows the combined gag and pro sequences as shown in FIGS. 1 and 3. The nucleotide sequence according to the present invention is shown in upper case. FIG. 10 shows additional gag sequence with the nucleocapsid region in lower case.

FIG. 21 shows the full determined HRV-5 sequence.

FIG. 22 shows the sequence of HRV-5 Gag-PR in an EcoRI digested pBlueScript KS+ vector.

Further, the present invention provides a nucleic acid molecule which has a nucleotide sequence encoding a polypeptide which includes the amino acid sequence shown in FIGS. 1, 2, 3, 6, 10, 11, 12, 13, 14, 15, 16, 18, 19, 20, or 21, or part thereof. FIG. 2 shows a comparison between the amino acid sequence shown in FIG. 1 (HRV-5) and that obtained from samples from seven individuals (SEQ ID NOS: 21, 22, 23, 24, 25, 26, 27, 28). As can be seen from the figure, natural variation in the HRV-5 amino acid sequence occurs between individuals.

The coding sequence may be that shown in FIGS. 1, 3, 6, 10, 11, 12, 13, 14, 15, 16, 18, 19, 20 or 21 or it may be a mutant, variant derivative or allele of these sequences. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code as shown, for example, in FIG. 2.

As used herein the term “variant” applies to retroviral sequences which are homologous in the gag, pol, protease, nucleocapsid, RT/RNaseH, integrase or dUTPase genes to the sequence shown in FIG. 1, FIG. 3, FIG. 6, FIG. 10, FIG. 12, FIG. 13, FIG. 14, FIG. 15, FIG. 16, FIG. 18, FIG. 19 or FIG. 21 for example having at least 80%, or at least 85% or at least 90%, preferably 95%, or even more preferably 98% homology to the sequence. The term “fragment” refers to fragments which are large enough to hybridise under stringent conditions to said sequence. Suitably such fragments will be from 20 bases to 1 kilobase in length, and preferably from 400-500 bases in length.

Generally, nucleic acid according to the present invention is provided as an isolate, in isolated and/or purified form, or free or substantially free of material with which it is naturally associated, such as free or substantially free of nucleic acid flanking the gene in the human genome. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding all or part of the protease, gag, pol, integrase, RT/RNaseH, nucleocapsid or dUTPase genes and/or its regulatory elements, such as the LTR, can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992). These techniques include the use of the polymerase chain reaction (PCR) to amplify samples of such nucleic acid, e.g. from genomic sources or chemical synthesis. Modifications to the HRV-5 sequences can be made, e.g. using site directed mutagenesis, to lead to the expression of modified HRV-5 polypeptides or to take account of codon preference in the host cells used to express the nucleic acid.

In order to obtain expression of the HRV-5 nucleic acid sequences, the sequences can be incorporated in a vector having control sequences operably linked to the HRV-5 nucleic acid to control its expression. Such sequences may optionally include the HRV5 LTR sequences disclosed herein. The vectors may include other sequences such as promoters or enhancers to drive the expression of the inserted nucleic acid, nucleic acid sequences so that the HRV-5 polypeptides are produced as a fusion and/or nucleic acid encoding secretion signals so that the polypeptides produced in the host cell are secreted from the cell. The particular polypeptides can then be obtained by transforming the vectors into host cells in which the vector is functional, culturing the host cells so that the polypeptides are produced and recovering the polypeptides from the host cells or the surrounding medium. Prokaryotic and eukaryotic cells are used for this purpose in the art, including strains of E. coli, yeast, and eukaryotic cells such as COS or CHO cells. The choice of host cell can be used to control the properties of the polypeptides expressed in those cells, e.g. controlling where the polypeptides are deposited in the host cells or affecting properties such as there glycosylation. The vectors and host cells described above each form separate aspects of the present invention.

PCR techniques for the amplification of nucleic acid are described in U.S. Pat. No. 4,683,195. In general, such techniques require that sequence information from the ends of the target sequence is known to allow suitable forward and reverse oligonucleotide primers to be designed to be identical or similar to the polynucleotide sequence that is the target for the amplification. PCR comprises steps of denaturation of template nucleic acid (if double-stranded), annealing of primer to target, and polymerization. The nucleic acid probed or used as template in the amplification reaction may be genomic DNA, cDNA or RNA. PCR can be used to amplify specific sequences from genomic DNA, specific RNA sequences and cDNA transcribed from mRNA, bacteriophage or plasmid sequences. The HRV-5 nucleic acid sequences provided herein readily allow the skilled person to design PCR primers. References for the general use of PCR techniques include Mullis et al, Cold Spring Harbor Symp. Quant. Biol., 51:263, (1987), Ehrlich (ed), PCR technology, Stockton Press, NY, 1989, Ehrlich et al, Science, 252:1643-1650, (1991), “PCR protocols; A Guide to Methods and Applications”, Eds. Innis et al, Academic Press, New York, (1990).

Also included within the scope of the invention are antisense oligonucleotide sequences based on the HRV-5 nucleic acid sequences described herein. Antisense oligonucleotides may be designed to hybridize to the complementary sequence of nucleic acid, interfering with the production of polypeptide encoded by a given DNA sequence, or simply the replicative and invasive processes of the retrovirus. The construction of antisense sequences and their use is described in Peyman and Ulman, Chemical Reviews, 90:543-584, (1990), Crooke, Ann. Rev. Pharmacol. Toxicol., 32:329-376, (1992), and Zamecnik and Stephenson, P.N.A.S, 75:280-284, (1974).

Oligonucleotide probes or primers, as well as the full-length sequence (and mutants, alleles, variants and derivatives) are also useful in screening a test sample containing nucleic acid for the presence of HRV-5, the probes hybridising with a target sequence from a sample obtained from the individual being tested. The conditions of the hybridisation can be controlled to minimise non-specific binding, and preferably stringent to moderately stringent hybridisation conditions are preferred. The skilled person is readily able to design such probes, label them and devise suitable conditions for the hybridisation reactions, assisted by textbooks such as Sambrook et al (1989) and Ausubel et al (1992).

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RNAase cleavage and allele specific oligonucleotide probing.

Probing may employ the standard Southern blotting technique. For instance DNA may be extracted from cells and digested with different restriction enzymes.

Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

Preliminary experiments may be performed by hybridizing under low stringency conditions various probes to Southern blots of DNA digested with restriction enzymes. Suitable conditions would be achieved when a large number of hybridizing fragments were obtained while the background hybridization was low. Using these conditions nucleic acid libraries, e.g. cDNA libraries representative of expressed sequences, may be searched.

Those skilled in the art are well able to employ suitable conditions of the desired stringency for selective hybridization, taking into account factors such as oligonucleotide length and base composition, temperature and so on. Generally, specific primers are upwards of 14 nucleotides in length, but not more than 18 to 24. Those skilled in the art are well versed in the design of primers for use processes such as PCR.

In accordance with the present invention, nucleic acids, e.g. probes or primers, having the appropriate level of sequence homology with the protein coding region of any of the nucleic acid sequences mentioned herein may be identified by using hybridization and washing conditions of appropriate stringency. For example, hybridizations may be performed, according to the method of Sambrook et al., (22) using a hybridization solution comprising: 5× SSC, 5× Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2× SSC and 1% SDS; (2) 15 minutes at room temperature in 2× SSC and 0.1% SDS; (3) 30 minutes-1 hour at 37° C. in 1× SSC and 1% SDS; (4) 2 hours at 42-65° C. in 1× SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

A further aspect of the present invention provides an oligonucleotide or polynucleotide fragment of the nucleotide sequence shown in FIGS. 1, 3, 6, 10, 11 or 13, 14, 15, 16, 17, 18, 19, 20, 21 or 22 or a complementary sequence, in particular for use in a method of obtaining and/or screening nucleic acid. The sequences referred to above may be modified by addition, substitution, insertion or deletion of one or more nucleotides, but preferably without abolition of ability to hybridise selectively with nucleic acid of HRV-5, that is wherein the degree of homology of the oligonucleotide or polynucleotide with one of the sequences given is sufficiently high.

In some preferred embodiments, oligonucleotides according to the present invention that are fragments of any of the sequences shown in FIGS. 1, 3 6, 10, 12 or 13-22 are at least about 10 nucleotides in length, more preferably at least about 15 nucleotides in length, more preferably at least about 20 nucleotides in length. Such fragments themselves individually represent aspects of the present invention. Fragments and other oligonucleotides may be used as primers or probes as discussed but may also be generated (e.g. by PCR) in methods concerned with determining the presence in a test sample of HRV-5.

Methods involving use of nucleic acid in diagnostic and/or prognostic contexts, for instance in determining the presence of HRV-5 are discussed below.

The present invention also provides polypeptides encoded by the nucleic acid sequences provided in FIGS. 1, 3, 6, 10, 12 or 13-16, 18-22.

The skilled person can use the techniques described herein and others well known in the art to produce large amounts of the nucleocapsid, dUTPase, protease, RT/RNase H and integrase polypeptides, or fragments or active portions thereof, for use as pharmaceuticals, in the developments of drugs and for further study into its properties and role in vivo. Further, also within the scope of the present invention are viral proteins such as superantigens or regulatory proteins which may be produced by HRV-5. Such proteins are usually found close to the 3′ end of the virus. All HRV-5 proteins and polypeptides or fragments thereof will have commercial value apparent to the skilled person, for example as antigens for vaccines, for raising virus-specific antibodies and also for serological assays such as ELISAs and western blots.

Thus, a further aspect of the present invention provides a polypeptide which has the amino acid sequence shown in FIGS. 1, 2, 3, 6, 10, 11 or 12, 14-16, 18-22 which may be in isolated and/or purified form, free or substantially free of material with which it is naturally associated, such as other polypeptides or such as human endogenous polypeptides other than HRV-5 polypeptide or (for example if produced by expression in a prokaryotic cell) lacking in native glycosylation, e.g. unglycosylated.

Polypeptides which are amino acid sequence variants, alleles, derivatives or mutants are also provided by the present invention. A polypeptide which is a variant, allele, derivative or mutant may have an amino acid sequence which differs from that given in FIGS. 1, 2, 3, 6, 10, 11, 12, 13-16, 18-22 by one or more of addition, substitution, deletion and insertion of one or more amino acids as shown in FIG. 2. Each variant shown in FIG. 2 and FIG. 20 forms a separate aspect of the invention. Preferred such polypeptides have HRV-5 function, that is to say have one or more of the following properties: immunological cross-reactivity with an antibody reactive with the polypeptide for which the sequence is given in FIGS. 1, 2, 3, 6 or 21; sharing an epitope with the polypeptide for which the amino acid sequence is shown in FIGS. 1, 2, 3, 6, 10, 11, 12, 13 or 21 (as determined for example by immunological cross-reactivity between the two polypeptides).

A polypeptide which is an amino acid sequence variant, allele, derivative or mutant of the amino acid sequence shown in FIGS. 1, 2, 3, 6, 10, 11, 12, 13 or 21 may comprise an amino acid sequence which shares greater than about 50% sequence identity with the sequence shown in FIGS. 1, 2, 3, 6, 10, 11, 12, 13 or 21 greater than about 60%, greater than about 70%, greater than about 75%, greater than about 80%, greater than about 85%, greater than about 90% or greater than about 95%. Particular amino acid sequence variants may differ from those shown in FIGS. 1, 2, 3, 6, 10, 11, 12 or 21by insertion, addition, substitution or deletion of 1 amino acid, 2, 3, 4, 5-10, 10-20 20-30, 30-50, 50-100, 100-150, or more than 150 amino acids. Examples of variants are shown in FIGS. 2 and 20 each form a separate aspect of the invention.

Screening for the presence of one or more of these in a test sample has a diagnostic and/or prognostic use, for instance in determining the presence of HRV-5, as discussed below. The present invention also includes active portions, fragments, derivatives of the HRV-5 polypeptides of the invention.

A “fragment” of an HRV-5 polypeptide means a stretch of amino acid residues of at least about five to seven contiguous amino acids, often at least about seven to nine contiguous amino acids, typically at least about nine to 13 contiguous amino acids and, most preferably, at least about 15, 20 to 30 or more contiguous amino acids. Fragments of an HRV-5 polypeptide sequence may comprise antigenic determinants or epitopes useful for raising antibodies to a portion of the amino acid sequences.

A polypeptide according to the present invention may be isolated and/or purified (e.g. using an antibody) for instance after production by expression from encoding nucleic acid. Polypeptides according to the present invention may also be generated wholly or partly by chemical synthesis. The isolated and/or purified polypeptide may be used in formulation of a composition, which may include at least one additional component, for example a pharmaceutical composition including a pharmaceutically acceptable excipient, vehicle or carrier. A composition including a polypeptide according to the invention may be used in prophylactic and/or therapeutic treatment as discussed below.

A polypeptide, peptide fragment, allele, mutant or variant according to the present invention may be used as an immunogen or otherwise in obtaining specific antibodies. Antibodies are useful in purification and other manipulation of polypeptides and peptides, diagnostic screening and therapeutic contexts. This is discussed further below.

A polypeptide according to the present invention may be used in screening for molecules which affect or modulate its activity or function. Such molecules may be useful in a therapeutic (possibly including prophylactic) context.

A further important use of the HRV-5 polypeptides is in raising antibodies, or at least antibody binding domains, that have the property of specifically binding to the HRV-5 polypeptides, or fragments or active portions thereof.

The production of monoclonal antibodies is well established in the art. Monoclonal antibodies can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-239400. A hybridoma producing a monoclonal antibody may be subject to genetic mutation or other changes, which may or may not alter the binding specificity of antibodies produced.

The provision of the novel HRV-5 polypeptides enables for the first time the production of antibodies able to bind it specifically. Accordingly, a further aspect of the present invention provides an antibody able to bind specifically to the polypeptide whose sequence is given in FIGS. 1, 2, 3, 6, 10, 11, 12, 13 or 21. Such an antibody may be specific in the sense of being able to distinguish between the polypeptide it is able to bind and other human endogenous polypeptides for which it has no or substantially no binding affinity (e.g. a binding affinity of about lOOOx worse). Specific antibodies bind an epitope on the molecule which is either not present or is not accessible on other molecules. Antibodies according to the present invention may be specific for the wild-type polypeptide. Antibodies according to the invention may be specific for a particular mutant, variant, allele or derivative polypeptide as between that molecule and the wild-type HRV-5 polypeptides, so as to be useful in diagnostic and prognostic methods as discussed below. Antibodies are also useful in purifying the polypeptide or polypeptides to which they bind, e.g. following production by recombinant expression from encoding nucleic acid.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunizing a mammal (e.g. mouse, rat, rabbit, horse, goat, sheep or monkey) with the protein or a fragment thereof. Antibodies may be obtained from immunized animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal.

As an alternative or supplement to immunizing a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunized with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Exemplary antibody fragments, capable of binding an antigen or other binding partner are the Fab fragment consisting of the VL, VH, C1 and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanized antibodies in which CDRs from a non-human source are grafted onto human framework regions, typically with the alteration of some of the framework amino acid residues, to provide antibodies which are less immunogenic than the parent non-human antibodies, are also included within the present invention

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the CDRs, of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The present invention also provides protein antigens obtained from the sequences provided herein. The protein antigens may be used in the preparation of vaccines. If the purified protein is not antigenic per se, it can be bound to a carrier to make the protein immunogenic. Carriers include bovine serum albumin, keyhole limpet hemocyanin and the like. Vaccination can be conducted in conventional fashion. For example, the antigen, whether a viral particle or a protein, can be used in a suitable diluent such as water, saline, buffered salines, complete or incomplete adjuvants. The immunogen may be administered using standard techniques for antibody induction, such as by subcutaneous administration of a physiologically compatible, sterile solutions containing inactivated or attenuated virus particles or antigens.

As a further aspect, the present invention provides agents for use in treatment, diagnosis and therapy of autoimmune and other inflammatory diseases such as arthritis and SLE associated with HRV-5.

The HRV-5 polypeptides, antibodies, peptides and nucleic acid of the invention described above as well as those derived from the nucleic acid and amino acid sequences shown in FIGS. 1, 2, 3, 6, 10, 11, 12, 13 or 21 can be formulated in pharmaceutical compositions. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes. The invention provides methods of treatment of autoimmune or other inflammatory diseases involving, for example, the application of inhibitors of retroviral replication such as inhibitors of reverse transcription (such chain terminators, for example zidovudine) and protease inhibitors or other anti-viral drugs. Further examples include inhibitors of integrase or dUTPase activity or inhibitors of accessory proteins such as regulatory proteins which may be produced by the virus.

For use in these methods, the above-mentioned agents may be suitably administered in the form of a pharmaceutical composition in which they are combined with a pharmaceutically acceptable carrier or diluent. Such compositions form a further aspect of the invention.

Suitable pharmaceutically acceptable carriers include solid and liquid carriers such as water, aqueous ethanol or the like, as are conventional in the art. The form of the composition may be suitable for oral, topical or parenteral use. Suitable forms of the composition include tablets, capsules, syringes, creams, suspensium, solutions, reconstitutable powders and sterile forms for injection or infusions. Other conventional pharmaceutical acceptable materials such as diluents, binders, preservative etc may be included.

The agent is administered in a therapeutically effective amount. The precise dosage will depend upon the particular agent being employed. The nature of the disease being located as well as the patient and can be determined by a clinician in the usual manner.

The agents may be capable of protecting a patient immunized therewith against infection or the consequence of infection by the corresponding wild-type virus.

With regard to diagnosis, wild-type virus may be detected in tissue samples using an assay system developed from knowledge of HRV-5. For example, the present invention provides the diagnosis of autoimmune and other inflammatory diseases, particularly arthritis, by use of a specific binding member such as (a) nucleic acid hybridizable with a nucleic acid associated with wild-type HRV-5; (b) a substance comprising an antibody binding domain with specificity for one or more epitopes or sequences characteristic of polypeptide expressed by the wild-type HRV-5.

Where the specific binding member comprises nucleic acid, the member may simply be used as a specific probe in accordance with standard techniques and procedures. Alternatively, the specific binding member may comprise a pair of oligo- or polynucleotide sequences for use in an amplification technique such as PCR.

In particular, the present invention provides oligonucleotide primer pairs for amplification of polynucleotide sequences (be they in the form of DNA, RNA, single-stranded or double-stranded) which comprises, or is derived from, the nucleotide sequence of HRV-5 as shown in FIGS. 1 to 6, 10 to 13 and 14-22.

The primer pairs may be designed by use of the sequence information provided herein. Having increased the copy number of polynucleotide sequence associated with HRV-5, the amplified sequences may be detected by standard methods such as the provision of radioactive nucleotides for inclusion in the sequences being copied, ethidium bromide staining, sequencing and hybridization probing.

The present invention therefore provides a method for diagnosing autoimmune diseases, particularly arthritis, by taking a suitable sample from a patient, for example, the synovial membrane, and detecting the presence or absence of HRV-5 by adding to the sample suitable specific binding members as described above. If the specific binding member was a pair of oligonucleotide primers, the method may also include the steps of adding other standard ingredients for carrying out a polynucleotide sequence amplification (an amplification based on a DNA template or an RNA template), and applying standard hybridization, elongation and denaturation or strand separation conditions to amplify any new polynucleotide sequence positioned between the two primers and looking for the presence or absence of an amplified product the determine the presence or absence of HRV-5.

Generally, as mentioned earlier, specific primers are upwards of 14 nucleotides in length, but not more than 18-24.

A further aspect of the present invention provides an oligonucleotide or polynucleotide fragment of the nucleotide sequences disclosed herein, or a complementary sequence, in particular for use in a method of obtaining and/or screening nucleic acid. The sequences referred to above may be modified by addition, substitution, insertion or deletion of one or more nucleotides, but preferably without abolition of ability to hybridize selectively with nucleic acid characteristic of HRV-5, that is wherein the degree of homology of the oligonucleotide or polynucleotide with one of the sequences given is sufficiently high.

Oligonucleotides according to the present invention that are fragments of any of the sequences disclosed herein are at least about 10 nucleotides in length, more preferably at least about 15 nucleotides in length, more preferably at least about 20 nucleotides in length. Such fragments themselves individually represent aspects of the present invention.

Preferably oligonucleotide primers according to the present invention comprise the nucleic acid sequence:

| | | |
|---|---|---|
| A | 5′ - TCAGAAGGTGATTGGCCGAAGTCA - 3′; | (SEQ ID NO: 29) |
| | 5′ - GGTCCTCATTTGTTAATGTCAGTC - 3′; | (SEQ ID NO: 30) |
| or | | |
| B | 5′ - CCCTTCAGCCAGGAGATAATACT - 3′; | (SEQ ID NO: 31) |
| | 5′ - ATGTCTCTTCCCCATAATGTGATG - 3′; | (SEQ ID NO: 32) |

Preferably the above two sets of primers are used in nested PCR assay with set A being first stage primers and set B being second set primers.

The present invention provides further primer sets for use in assays described above, comprising the nucleic acid sequence:

| | |
|---|---|
| C | 5′ - CCATCACATTATGGGGAAGAGACA - 3′; (SEQ ID NO: 33) |
| | 5′ - GAATGTCTTGTTCATGTAGAGGTAT - 3′; (SEQ ID NO: 34) |
| D | 5′ - GCCATTGTCATGGCTGGACAACAA - 3′; (SEQ ID NO: 35) |
| | 5′ - CCTTCAGATCGAGTACTATTAATGG - 3′; (SEQ ID NO: 36) |

wherein set C may be used as first stage primers and set D may be used as second set primers.

| | | |
|---|---|---|
| E | 5′ - GCCATGACACCATCAAGAAGTGCT - 3′; | (SEQ ID NO: 37) |
| | 5′ - TGCTTTGGGATCATAGTAGGAAC - 3′; | (SEQ ID NO: 38) |
| F | 5′ - ATTAGGCTCCAGAGAAGGCAGAAG - 3′; | (SEQ ID NO: 39) |
| | 5′ - CCGGGAGTCCAGGTTGTAATG - 3′; | (SEQ ID NO: 40) |

wherein set E may be used as first stage primers and set F may be used as second set primers.

Using a nested PCR technique, the applicants have found that samples containing as little as 1-10 molecules of viral DNA per sample can be detected. When using this technique, care should be taken with controls in order to avoid false positives.

The above sequences may also be used to design longer nucleotide probes useful in detecting HRV-5 sequences. Additional nucleotide bases (e.g. N=0 to 200 where N may be any nucleotide) may be added to either end of these primers. Preferably, these additional nucleotide bases are derived from the sequence shown in FIG. 12 or 21 (SEQ ID NOS: 15, 18 and 127).

Diagnostic tests of infection by the virus based on immunological methods, such as peptide and protein enzyme linked immunosorbent assay (ELISAs), and western blots, as well as on PCR and other DNA or RNA detection methods, form a further aspect of the invention.

As described above, viral antigens form a further aspect of the invention. For instance, the retrovirus or viral antigens can be used to raise antibodies which may be monoclonal or polyclonal in a conventional manner.

These antibodies can be used to screen samples such as synovial membrane samples and other tissues or cell cultures (e.g. peripheral blood cells) taken from patients suspected of suffering from, for example, arthritis and other diseases, by for example immunohistochemistry, for the presence of virus. Therefore the invention also provides an antibody which binds an antigen of the above described as well as diagnostic kits which contain said antibody.

Further suitable antigens which can be used to raise antibodies are those containing epitopes from the matrix (MA) and capsid (CA) and other gag proteins as well as env, pro, pol, dUTPase, RT/RNase H, integrase, viral regulatory proteins or superantigens.

Viral antigens according to the present invention may be used to raise an immune response in a mammalian subject, preferably a human subject and as such be used in the production of vaccines. Conventional vaccines comprise either infectious (“live”) or non-infectious (“killed”) virus particles. Upon administration, all vaccines should have the following properties: a) cause less severe disease than the natural infection; b) stimulate effective and long-lasting immunity, and c) be genetically stable. The production of vaccines is now well developed in the art. With killed virus vaccines, it can be a problem to produce sufficient material cheaply and ensure that no infectious virus survives the inactivation procedure. Therefore, DNA technology may be used to identify parts of the viral genome that encodes particular viral proteins against which protective immunity may be directed. This may be achieved by a)expression of the entire protein, e.g. GAG, CA, or ENV, or any other viral proteins, particularly regulatory proteins; expression of a fragment of the protein containing the antigenic site; or c) chemical synthesis of a peptide which contains the antigenic site. For a) and b) viral nucleic acid (e.g. FIGS. 1, 3, 6, 10, 12, 13 or 21 variants or fragments thereof) may be excised and inserted into an appropriate expression vector, together with control (promoter, stop and polyadenylation) signals. In this way there is a small part of the viral genome, by definition non-infectious, which by insertion into a host cell growing on an industrial scale will produce very large amounts of protein very cheaply. Bacterial expression systems may be used. However, in order to accomplish eukaryotic-type cotranslational and post-translational modifications, such as glycosylation and proteolytic cleavage, eukaryotic cells may be used.

Live vaccines evoke the most effective immunity and therefore a nucleotide sequence encoding the antigenic site of interest may be inserted into a pre-existing live virus, so that it is expressed naturally as the virus multiplies. This has previously been achieved for viruses including influenza, rabies, herpes simplex type I and hepatitis B viruses, using vaccinia virus as the live vaccine.

The present invention provides vaccines comprising nucleotide sequences or polypeptide sequences as disclosed above. Further, the present invention provides methods of treating a mammalian subject using such vaccines so as to raise an immune response.

In addition, the invention provides methods of detecting antibodies to viral proteins. These methods are useful in disease diagnosis, including RA, systemic lupus erythematosus and other autoimmune diseases, for example, see Table I.

Particularly preferred is an ELISA for detection of antibodies to the virus peptides or proteins. Specific assay devices of the invention comprise a viral antigen of the retrovirus of the invention immobilized on a support. Suitably purified recombinant viral antigens are used. These antigens may be expressed in eukaryotic or prokaryotic cells such as bacterial, yeast or mammalian cells, preferably bacterial cells. Affinity purified anti-viral antigen sera such as rabbit sera can be used to capture antigen for immobilization.

In addition cultured virus could form the basis of a virus isolation assay as is known in the art. Methods which may be useful in the culture of the virus include direct culture methods (such as those described by Weiss R. A., Chpt 3 in Weiss et al (eds), 1982 RNA Tumor Viruses (Cold Spring Harbor Laboratory press) and Brookes et al., Brit. J. Rheum. 1995 34: 226-231), co-cultivation methods, for example by culturing tissue samples with a target cell line. In such a method, the tissue samples is either digested with trypsin or homogenized with a mortar and pestle. This is then placed into a flask with typically 105-106 tissue culture cells.

Suitable tissue cells which are permissive for viral growth may include T and B lymphocytes, monocytes, macrophages, fibroblasts and epithelial cells.

Alternatively, virus may be cultured by xenografting virus into suitably nude or severe combined immunodeficient (SCID) mice. Using this method, tissue samples such as synovial membrane, may be implanted subcutaneously for example into the mid-flank of an anaesthetized mouse. After this, evidence of virus growth may be assessed using PCR for RNA and/or DNA or by the sensitive RT assay described by Silver et al., Nucleic Acids Res. 1993, 21: 3593-3594, and the virus isolated.

The association of HRV-5 with autoimmune diseases and inflammatory bowel diseases as discussed above, allows for screening methods to determine agents such as chemical compounds which are effective in the treatment of these diseases. Such screening methods, together with agents discovered as a result of them, form a further aspect of the invention.

The present invention further provides a vector for use in gene therapy comprising disabled HRV-5. Vectors such as viral vectors have been used in the prior art to introduce gene into a wide variety of different target cells. Typically the cells are exposed to the target cells so that transfection can take place in a sufficient proportion of the cells to provide a useful therapeutic or prophylatic effect from expression of the desired polypeptide. The transfected nucleic acid may be permanently incorporated into the genome of each of the targeted cells, providing long lasting effect, or alternatively, the treatment may have to be repeated periodically. Disabled HRV-5 virus vectors may be prepared by deletion or inactivation of one or more specific viral proteins, by standard methods apparent to the skilled person (Naldini zufferey R. et al Nature Biotechnol. 1997 15:871-875).

Such vectors may be utilized to deliver nucleic acids encoding therapeutic molecules to tissues which are selectively infected by HRV-5. In this embodiment, the LTR of the virus is ligated upstream of a therapeutic gene of interest. A helper virus is co-administered with this construct to a target cell in vitro. Such helper viruses enable replication of defective viruses by providing structural proteins and enzymes in trans during the mixed infection and thereby generate viral particles capable of a single round of infection. A suitable helper virus allows LTR-gene constructs that lack almost all the viral genome (which has been replaced with the therapeutic gene of interest) to infect target cells when subsequently administered in vivo.

Specific transgenes or therapeutic genes for use in the vectors of the invention can fall into several categories. The identity of such genes and their GenBank Accession numbers are provided below. These include, without limitation, cytokines (e.g., interleukin-2 (GenBank Accession code U25676), and IL-7 (XM_{—}005266)), tumor suppressor genes (e.g., p53 (P04637) and suicide′genes such as herpes simplex virus type 1 thymidine kinase (V00470). Other transgenes may be functional copies of a single gene where the disease is due to a defective or mutated copy of this gene. Example of such diseases (and the therapeutic gene) are adenosine deaminase deficiency (adenosine deaminase, NM_{—}000022), mucopolysaccharidosis type II (iduronate-2-sulfatase, XM_{—}018134), and familial hypercholesterolemia (low density lipoprotein receptor, NM_{—}000527) and cystic fibrosis (cystic fibrosis transmembrane conductance regulator, M28668).

The ability of HRV-5 to infect synovial membrane may confer unique properties on HRV-5 derived vectors as gene vectors for treatment of rheumatoid arthritis. Suitable transgenes in this case may be cytokines or related immunomodulatory molecules such as the interleukin-1 receptor antagonist protein (IRAP, GenBank code NM_{—}000577).

The vectors described above will also be useful for the generation of cell lines and/or transgenic animals expressing heterologous genes of interest.

### BRIEF DESCRIPTION OF THE DRAWINGS

Aspects and embodiments of the invention will now be described, by way of example, with reference to the accompanying figures. Further aspects and embodiments will be apparent to those skilled in the art. All documents mentioned in this text are incorporated herein by reference.

FIG. 1 shows the nucleotide sequence (SEQ ID NOS: 1 and 4) and derived amino acid sequence of HRV-5 nucleocapsid and dUTPase coding regions (SEQ ID NOS: 2 and 3). Both strands of the nucleotide sequence of the new sequence are shown with the deduced amino acid sequences of the gag and pro genes. The previously published region is shown in lower case. Nucleotides 1-26 are derived from the degenerate primer (8532) and so may not represent the genuine sequence of HRV-5 at these positions. Nucleotides 884-907 represent specific primer 4146 which was also used to amplify this region.

FIG. 2 shows an alignment of the deduced nucleocapsid amino acid sequences of HRV-5 from seven individuals (SEQ ID NOS: 22-29). The sequences are shown aligned with the prototype HRV-5 clone. A dash (-) indicates identity with HRV-5 and a dot (.) indicates a gap introduced into the alignment to allow for insertions and deletions. The sequence marked NC20 was amplified from DNA from the blood of a patient with rheumatoid arthritis. The other sequences (NC2-NC11) were amplified from DNA from the blood of patients with SLE.

FIG. 3 shows the sequence of the HRV-5 pol gene.

Both strands of DNA are shown (SEQ ID NOS: 5 and 7). The region of HRV-5 pol downstream of the previously published sequence is shown. The previously published sequence is shown in lower case. Note that nucleotides 1877-1896 are derived from the degenerate integrase primer used to clone this fragment and so may not represent the genuine sequence of the virus in this region.

FIG. 4 shows the deduced amino acid sequence of a DNA sequence used as a sequence tag and designated Sjo-1 and its alignment with other, known retroviral sequences (SEQ ID NOS: 41 and 48).

FIG. 5 is a nucleotide sequence and translation of JC96 showing two open reading frames (SEQ ID NOS: 49-52). The protease (PR) open reading frame is frame a and the reverse transcriptase (RT) open reading frame is frame c.

FIG. 6 shows the sequence data of FIGS. 1, 2 and 3 combined (SEQ ID NOS: 8, 9, 10 and 11). The novel sequence shown in upper case.

FIG. 7 shows alignments of deduced PR and RT amino-acid sequences of clones of JC96 from five individuals (SEQ ID NOS: 53-57). Note that the JC96 sequence extends further 5′ than the other clones. This is because JC96 was obtained using degenerate primers whereas the other clones were generated using internal specific primers based on the JC96 sequence.

FIG. 8 shows a diagram of Indirect ELISA using His-myc tagged proteins.

FIG. 9 shows a diagram of Capture ELISA using His-myc tagged proteins.

FIG. 10 shows sequence of a fragment of the HRV-5 Gag gene (SEQ ID NOS: 12 and 14). The nucleocapsid region (described in Example 3 and FIG. 1) is shown in lower case. The remaining sequence was cloned in 5 stages. The region between the Eco RI site (at nucleotide 1205, marked in bold) and the nucleocapsid was cloned by Vectorette PCR. The region between the Cla I site (nucleotide 262, marked in bold) and the Eco RI site was cloned by a separate Vectorette PCR. Nucleotides 1 to 461 were cloned in 3 stages using the rapid amplification of cDNA ends (RACE) method adapted for use on double stranded genomic DNA.

FIG. 11 shows CLUSTALW multiple sequence alignment of HRV-5 CA protein with CA proteins of other B and D-type retroviruses (SEQ ID NOS: 58, 59, 60 and 61). It should be noted that there is generally very little primary sequence similarity in this protein between different retroviruses. The most conserved region is known as the major homology region (MHR, Craven et al, 1995, J. Virology, 69: 4213-4227) and is indicated in bold typeface. Conserved residues and conservative substitutions are indicated by * and : respectively.

FIG. 12 shows the full HRV-5 sequence (gag-pro-pol; SEQ ID NOS: 13, 15, 16, 17, and 18).

FIG. 13 shows the sequence of the deposited HRV5gagpol 17.1 clone aligned with the consensus HRV-5 sequence from FIG. 12 (SEQ ID NOS: 19 and 20). Matches between the 2 sequences are marked with (|), gaps are indicated by a dash (-).

FIG. 14 shows a nucleic acid sequence of the Gag3fragment (SEQ ID NO: 92) with the deduced amino acid sequence (SEQ ID NO: 93). Only the coding (+) strand is shown. Nucleotides in lower case were also present in a previously cloned fragment.

FIG. 15 shows a nucleic sequence of the Gag4 fragment (SEQ ID NO: 94) with deduced amino acid sequence (SEQ ID NO: 95). Only the coding (+) strand is shown. Nucleotides in lower case were also present in the Gag3 fragment.

FIG. 16 shows a nucleic acid sequence of the Gag5fragment (SEQ ID NO: 96) with deduced amino acid sequence (SEQ ID NO: 97). Only the coding (+) strand is shown. Nucleotides in lower case were also present in the Gag4 fragment.

FIG. 17 shows a nucleic acid sequence of the Gag6 fragment (SEQ ID NO: 98). Only the forward (+) strand is shown. Nucleotides in lower case were also present in the Gag5 fragment.

FIG. 18 shows a nucleic acid sequence of the IN2 fragment (SEQ ID NO: 99) with deduced amino acid sequence (SEQ ID NO: 100). Only the coding (+) strand is shown. Nucleotides in lower case were also present in a previously cloned fragment.

FIG. 19 shows a nucleic acid sequence of the IN3 fragment (SEQ ID NO: 101) with deduced amino acid sequence (SEQ ID NO: 102). Only the coding (+) strand is shown. Nucleotides in lower case were also present in the IN2 fragment. Nucleotides in lower case and italics are not HRV-5 but are human DNA present downstream of the integration site.

FIG. 20 shows an alignment of the Gag-PR region of the reference strain with another clone obtained from the same patient.

FIG. 21 shows the nucleotide sequence of a representative clone of HRV-5 is shown (GenBank Accession AF******; SEQ ID NO: 127). This clone was assembled from several PCR fragments. Open reading frames for gag, pro and pol are shown (SEQ ID NO: 103) and potential heptanucleotide frameshifting sites between the ORFs are boxed. The putative PBSHIS and polypurine tract are marked in bold as are specific amino acid motifs discussed in the text (PPXY in gag, DTG in PR and YMDD in RT).

FIG. 22 shows the sequence of an HRV-5 Gag-PR product in a EcoRI digested pBlueScript KS+ vector (SEQ ID NO: 104).

FIGS. 23A and 23B are a series of micrographs and Western blots. Retroviral Gag proteins were tagged with green fluorescent protein (GFP) and expressed in 293T cells. FIG. 23A: Gag-GFP produced in 293T cells gives a cytoplasmic speckled pattern with HRV5 which is similar to that obtained for MPMV and unlike the cell surface staining seen with MLV Gag. FIG. 23B: Extracts from the cells shown in FIG. 23A were fractionated by sucrose density gradient centrifugation. As with MLV and MPMV, HRV-5 appears to form core particles with a density around 1.22 g/ml, the typical density of retroviral cores. Therefore, HRV-5 behaves as expected for a B/D type retrovirus in this system. Arrows indicate expected size of Gag-GFP fusion proteins (approx. 90 kDa).

### DETAILED DESCRIPTION OF THE INVENTION

### EXAMPLE 1

Development of Specific PCR Assays for HRV-5.

Three nested PCR assays specific for HRV-5 were developed. These assays each used primer sets derived from different regions of JC96 (Griffiths et al., 1997 J. Virol. vol. 71 pp. 2866-2872). Optimal PCR conditions and the sensitivity of each primer set were determined using a plasmid which contained the cloned sequence serially diluted in the presence of 500 ng of human genomic DNA. Under optimal conditions, all primer sets were found to have a sensitivity of nominally 1 molecule of viral DNA. Since 500 ng of human DNA represents the DNA content of approximately 75,000 cells, these primer sets should each be sufficiently sensitive to detect 1provirus in 75,000 cells.

The primer sets were:

Assay 1

First stage primers:

4143 5′- TCAGAAGGTGATTGGCCGAAGTCA-3′; (SEQ ID NO: 29)

4144 5′-GGTCCTCATTTGTTAATGTCAGTC-3′; (SEQ ID NO: 30)

Conditions: initial denaturation at 94° C., 4 mins followed by 40 cycles of 94° C., 45 secs; 52° C., 45 secs; 72° C., 45secs. One microlitre transferred to second stage.

Second stage primers:

4145 5′-CCCTTCAGCCAGGAGATAATACT-3′; (SEQ ID NO: 31)

4146 5′-ATGTCTCTTCCCCATAATGTGATG-3′; (SEQ ID NO: 32)

Conditions as first stage but for only 30 cycles.

Assay 2

First stage primers:

3493 5′-CCATCACATTATGGGGAAGAGACA; (SEQ ID NO: 33)

3496 5′-GAATGTCTTGTTCATGTAGAGGTAT; (SEQ ID NO: 34)

Conditions: initial denaturation at 94° C., 3 mins followed by 40 cycles of 94° C., 45 secs; 52° C., 45 seas; 72° C., 30 seas. One microliter transferred to second stage.

Second stage primers:

3494 5′- GCCATTGTCATGGCTGGACAACAA; (SEQ ID NO: 35)

3495 5′- CCTTCAGATCGAGTACTATTAATGG; (SEQ ID NO: 36)

Conditions as first stage but for only 30 cycles.

Assay 3

First stage primers:

0831 5′-GCCATGACACCATCAAGAAGTGCT; (SEQ ID NO: 37)

2061 5′-TGCTTTGGGATCATAGTAGGAAC; (SEQ ID NO: 38)

Conditions: initial denaturation at 94° C., 3 mins followed by 25 cycles of 94° C., 30 seas; 60° C., 60 seas; 72° C., 30seas. One microlitre transferred to second stage.

Second stage primers:

2062 5′-ATTAGGCTCCAGAGAAGGCAGAAG; (SEQ ID NO: 39)

2063 5′-CCGGGAGTCCAGGTTGTAATG; (SEQ ID NO: 40)

Conditions: initial denaturation at 94° C., 3 mins followed by 25 cycles of 94° C., 45 seas; 58° C., 60 seas; 72° C., 30 secs.

For each PCR one fifth of the reaction products were analysed by agarose gel electrophoresis.

### EXAMPLE 2

Detection of HRV-5 in Inflamed Joints

In preliminary experiments, each PCR assay was used to test a number of human DNA samples. Although the different PCR assays had similar sensitivities, surprisingly, primer set 1 (SEQ ID NOS: 29, 30) was found to detect HRV-5 sequences in more human DNA samples than did the other primer sets, i.e. many DNA samples found to be positive by assay 1 were negative using assays 2 and 3. This indicated that assay 1 is more sensitive for detecting HRV-5 DNA in clinical tissue samples than the other assays. This primer set (SEQ ID NOS: 29 and 30) was therefore used to screen a larger number of human DNA samples from a variety of tissues and diseases (Table 1).

TABLE 1
Frequency of detection of HRV-5 proviral DNA in
different tissue samples.

| | | Samples | Samples |
|---|---|---|---|
| Tissue | Disease | tested | positive |
| Synovium | Rheumatoid arthritis | 25 | 12 |
| | reactive arthritis | 5 | 3 |
| | Osteoarthritis | 5 | 3 |
| | Psoriatic arthritis | 2 | 2 |
| | Ankylosing spondylitis | 1 | 0 |
| | Normal | 7 | 0 |
| Salivary | Sjögren's syndrome | 26 | 0 |
| gland | Normal | 4 | 0 |
| Lymph node | | 27 | 0 |
| Non-malignant | | | |
| Bone marrow | Miscellaneous | 31 | 0 |
| Blood | Rheumatoid arthritis | 26 | 3 |
| | SLE | 56 | 11 |
| | Osteoarthritis | 3 | 1 |
| | Normal | 67 | 1 |
| Bowel | Crohn's disease | 10 | 1 |
| | Ulcerative colitis | 9 | 8 |

Of 38 synovial membranes studied from patients with various arthropathies, 20 were positive for HRV-5 proviral DNA (53%). Positive samples were identified from patients with rheumatoid arthritis, osteoarthritis and psoriatic arthritis. Seven normal synovial membranes were negative. In addition, DNA from 27 benign lymph nodes, 26 salivary gland biopsies from patients with primary Sjogren's syndrome, 4 normal salivary glands and 31 bone marrow biopsies were negative. Of 152 peripheral blood DNA samples tested, 16 (8%) were positive (3/26 rheumatoid arthritis, 11/56 systemic lupus erythematosus (SLE), 1/3 osteoarthritis and 1/67 normal blood).

The results of this PCR screen therefore indicate that HRV-5 can rarely be detected in most human DNAs, reaching a level of 1-2% in normal blood. Exceptions are in inflamed synovia where the rate of detection is >50% and in blood of patients with joint diseases and SLE.

### EXAMPLE 3

Cloning of the Nucleocapsid Region of HRV-5.

DNA samples found to be positive for HRV-5 sequences were used to amplify a region upstream of the known sequence of the virus. This PCR utilised a degenerate primer based on the zinc finger sequence motif conserved among retroviral nucleocapsid proteins. This degenerate primer was used in a hemi-nested PCR with reverse primers (from Assay 1) specific for the protease region of HRV-5. Due to the limited amounts of DNA in the samples available for study and the low abundance of HRV-5 in these DNA samples, DNA from different sources was pooled in order to increase the amount of target HRV-5 DNA in the PCR and thereby increase the chances of a successful amplification. The sources of the DNA were normal blood from an apparently normal subject and salivary gland DNA from a patient with rheumatoid arthritis.

The primers used were:

| | | |
|---|---|---|
| 8532 | 5′- TGYTTYAARTGYGGIMRIMMIGGICA; | (SEQ ID |
| | | NO: 62) |
| 4144 | 5′- GGTCCTCATTTGTTAATGTCAGTC; | (SEQ ID |
| | | NO: 30) |
| 4146 | 5′- ATGTCTCTTCCCCATAATGTGATG; | (SEQ ID |
| | | NO: 32) |

(where Y=C or T, R=A or G, M=A or C and I=inosine)

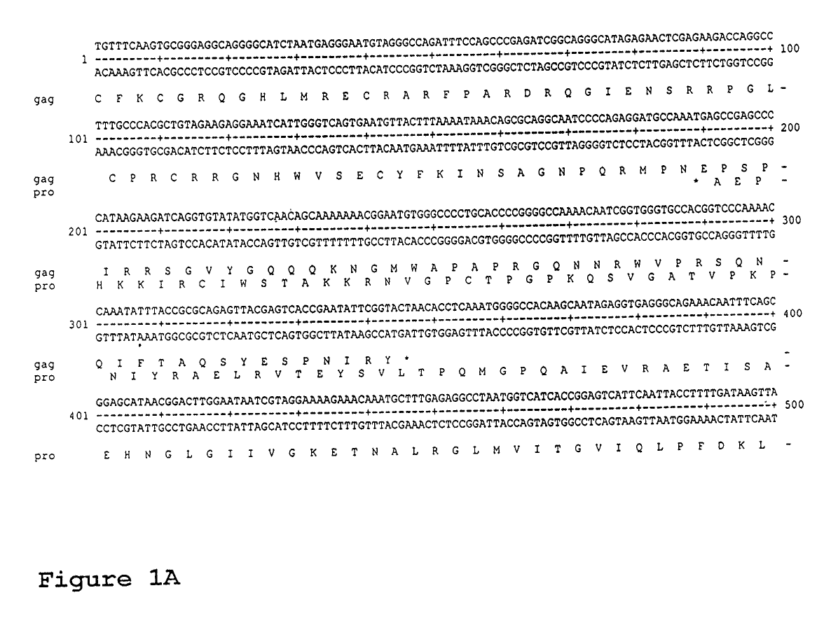

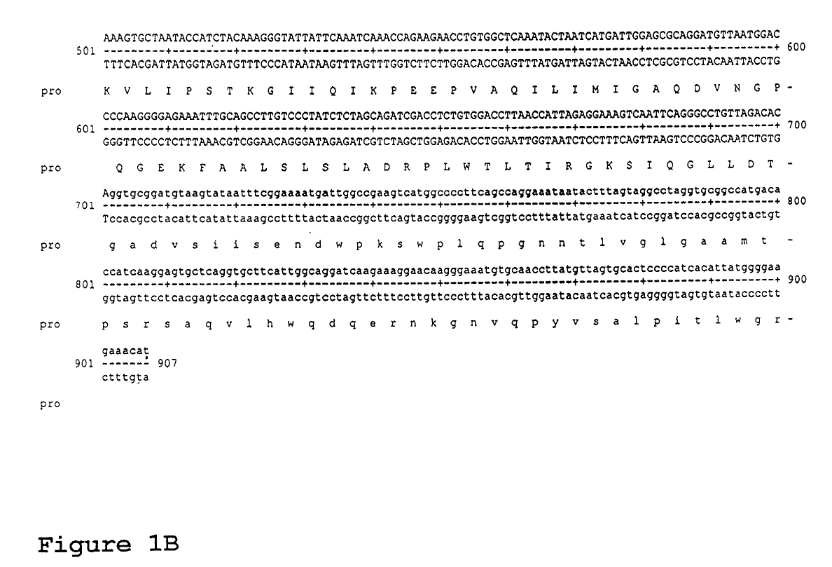

Approximately 1 μg of genomic DNA from each source were added to a 50 μl PCR reaction containing 10 mM Tris-Cl pH 8.3, 50 mM KCl, 2 mM MgCl2, 200 μM each dNTP, 2.5 units Taq polymerase (Qiagen) and 20 pmol of primers 8532 and 4144. The reactions were amplified on a Stratagene Robocycler Thermal cycler for 40 cycles of 94° C. 1 min, 42° C. 1 min 30 secs, 72° C. 3 mins with an initial denaturation at 94° C. for 3 mins. Three microliters of the products of this PCR were then reamplified using primers 8532 and 4146 for 40 cycles of 94° C. 1 min 10 secs, 42° C. 1 min 10 secs, 72° C. 3 mins with an initial denaturation at 94° C. for 3 mins. The products of this PCR were cloned into pBluescript using standard methods. Several plasmid clones were sequenced and a consensus found (FIG. 1).

### EXAMPLE 4

Amplification of HRV-5 Nucleocapsid Sequences from Patients with RA and SLE

DNA from patients with RA and SLE was tested for the presence of HRV-5 nucleocapsid sequences using nested PCR with primers specific for this region of HRV-5.

In the first stage, PCR, DNA was amplified with primers:

NCF3 5′-GCAGGGGCATCTAATGAGGGAAT-3′; (SEQ ID NO: 63)

NCR1 5′-CTGAAATTGTTTCYGCCCTCACCT-3′; (SEQ ID NO: 64)

wherein Y is a C or a T.

Conditions: initial denaturation at 94° C., 4 mins followed by 40 cycles of 94° C., 45 secs, 60° C., 45 secs; 72° C., 45 secs. One microliter of the products transferred to second stage.

Second stage primers:

| | | |
|---|---|---|
| NCF4 | 5′ - AGATTTCCAGCCCGAGATCGGCAG -3′; | (SEQ ID |
| | | NO: 65) |
| NCR2 | 5′ - TGTGGCCCCATTTGAGGTGTTAG -3′; | (SEQ ID |
| | | NO: 66) |

Conditions at first stage but for only 30 cycles.

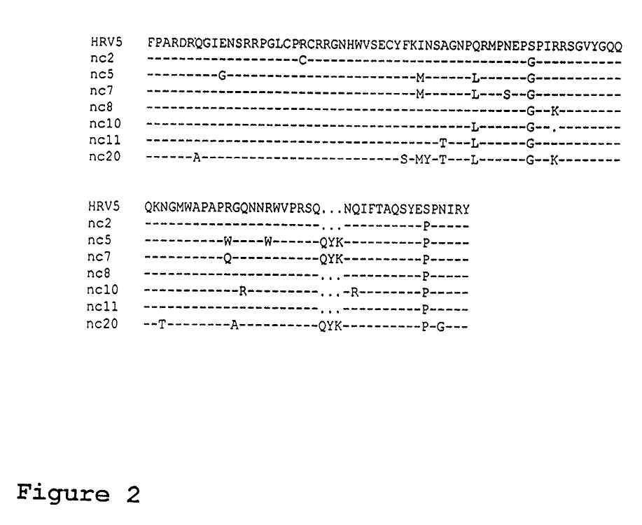

Following agarose gel electrophoresis, PCR products were purified, subcloned into pBluescript and several clones from each patient were sequenced. The variation in amino acid sequence is shown in FIG. 2.

### EXAMPLE 5

Cloning a Region of HRV-5 Integrase.

Following the successful amplification of a region upstream of HRV-5 protease, attempts were made to clone a region downstream of the previously known sequence. The DNA used in this experiment was from the blood of an apparently normal subject (same sample as above).

The primers used were:

| | | |
|---|---|---|
| 4143 | 5′-TCAGAAGGTGATTGGCCGAAGTCA | (SEQ ID NO: 29) |
| 3494 | 5′-GCCATTGTCATGGCTGGACAACAA | (SEQ ID NO: 35) |
| 3382 | 5′-CCAGGICCRTTRTCTGTTTT | (SEQ ID NO: 67) |
| 3383 | 5′-TGIGTRACATCCATTTGCCA | (SEQ ID NO: 68) |

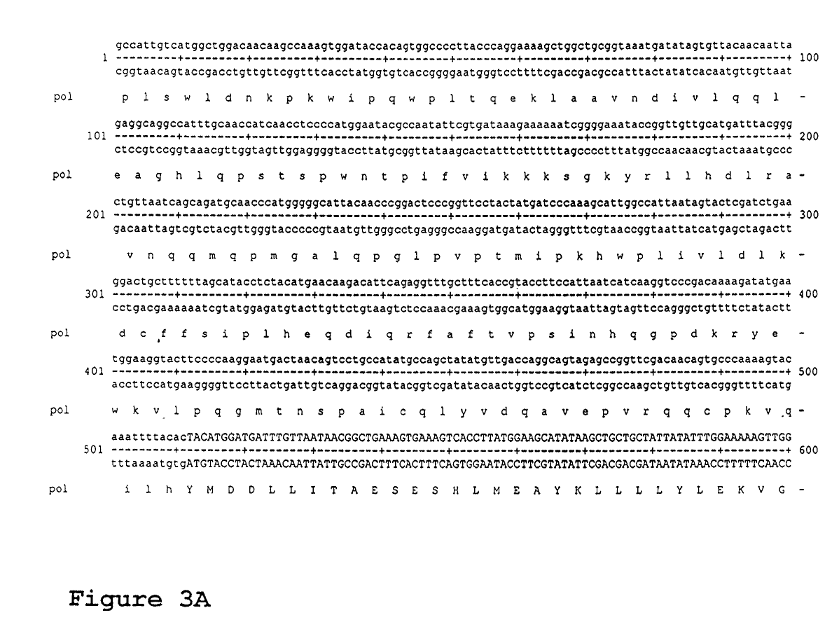

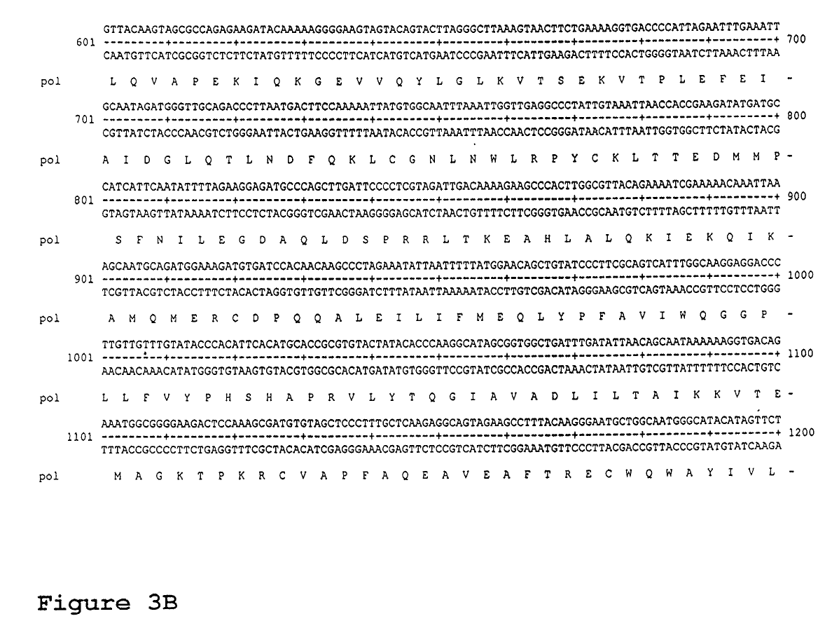

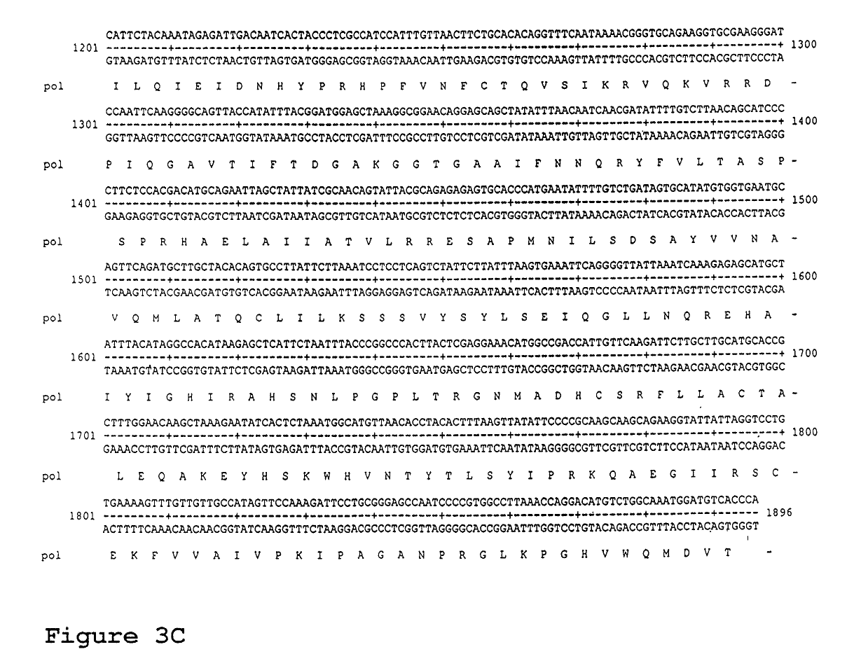

Approximately 3 μg of DNA were added to a 50 μl PCR reaction containing 20 pmol each of primers 4143 and 3382. The reactions were amplified on a Stratagene Robocycler Thermal cycler for 40 cycles of 94° C. 1 min 20 sec, 50° C. 1 min 30 secs, 72° C. 3 mins with an initial denaturation at 94° C. for 4 mins. One microliter of the products of this PCR was then reamplified using primers 3494 and 3383 for 40 cycles of 94° C. 1 min 20 secs, 44° C. lmin 30 secs, 72° C. 3 mins with an initial denaturation at 94° C. for 4 mins. The products of this PCR were cloned into pBluescript using standard methods. Several plasmid clones were sequenced and a consensus found (FIG. 3).

### EXAMPLE 6

Comparison of HRV-5 with other Described Retroviruses.

The conclusion from this comparison is that DNA from cells expressing HIAP-I and HIAP-II particles do not contain HRV-5 DNA.

In order to test whether the HIAP-I and HIAP-II cell lines described by Garry et al (Garry. R. F.; et al 1990Science 250:1127-1129; and Garry R. F.; et al Aids Res. Hum. Retroviruses 1996; 12: 931-940 repectively) contain HRV-5 sequences the two cell lines were obtained from the American Type Culture Collection. These cell lines have Accession Codes CRL-11213 (HIAP-I) and CRL-11622(HIAP-II). A vial of purified HIAP-II virus (VR-2503) was also obtained. The HIAP-I (VR-2394) virus preparation has not been released for study.

The cells were supplied as frozen ampules (on dry ice) and on receipt were immediately transferred to liquid nitrogen for storage. Subsequently, each vial was thawed at 37° C. and half of each cell stock was diluted in 10 ml RPMI-1640 culture medium supplemented with 10% foetal calf serum. The cells were then centrifuged at 1200 g for 5 mins at room temperature (22° C.) and the cell pellet suspended in 5 ml RPMI-1640 medium with 20% serum.

Cells were then cultured at 37° C. in a humidified atmosphere containing 5% CO₂.

The remaining half of each cell stock was then used to prepare DNA (using standard procedures) directly, without culture. This DNA was then tested for HRV-5 sequences using PCR Assay 1 and both cell lines were negative although PCR for control genomic sequnces were positive. This result demonstrates that the cell lines described by Garry et al. do not contain HRV-5 and therefore proves that HRV-5 is not the same virus as either HIAP-I or HIAP-II.

### EXAMPLE 7

Detection of HRV-5 DNA in Ulcerative Colitis.

HRV-5 specific PCR assay 1 (Example 1) was used to examine DNA from bowel biopsies from patients with Crohn's disease and ulcerative colitis. Of 10 bowel biopsies from Crohn's disease, HRV-5 DNA was detected in only 1 sample. In contrast, HRV-5 DNA was found in gut tissue from 8 of 9 patients with ulcerative colitis. Furthermore, the load of HRV-5 DNA in these samples was very high compared to that observed in other positive samples. One sample in particular had a sufficiently high load of HRV-5 DNA to permit the cloning of a further 1200 bp of the viral Gag gene (see Example 8).

### EXAMPLE 8

Cloning of a Region of the Gag Gene of HRV-5

Following the cloning of the nucleocapsid and integrase genes of HRV-5, attempts were made to clone additional regions of the Gag gene. Initial experiments which adopted the degenerate primer PCR approach were unsuccessful. Therefore, more general PCR strategies for cloning flanking DNA, or “chromosome walking”, were utilised. Specifically, the Vectorette PCR system [Arnold and Hodgson, 1991, PCR Methods Appl. 1: 39-42] was used successfully to clone 1.2 kbp of the HRV-5 Gag gene.

The Vectorette PCR system involves restriction enzyme digestion of the target DNA and subsequent ligation of double stranded oligonucleotide linkers (‘Vectorettes’) to the cut DNA ends. In practice, the target DNA is digested (separately) with a number of different enzymes in order to maximize the probability that one of these restriction sites is within a suitable range for PCR amplification. In addition, the oligonucleotide linker is designed in such a way that non-specific amplifications are minimized [Arnold and Hodgson, 1991, PCR Methods Appl. 1: 39-42].

For cloning the HRV-5 Gag gene, Vectorette PCR was performed using a kit obtained from Genosys Biotechnologies (UK). Five microgram aliquots of DNA from colon tissue of a patient with ulcerative colitis which was known to be positive for HRV-5 (using specific assay 1) were digested (separately) with the restriction enzymes Cla I, Eco RI, Bam HI and Hind III and the appropriate Vectorette linkers were ligated on to the cut DNA. Three rounds of digestion and ligation were performed to minimise concatamer formation as recommended in the manufacturer's protocol. Nested PCR was then performed on aliquots of the ligation products using HRV-5 specific primers in conjunction with primers derived from the linker sequence. This experiment resulted in the amplification of sequences upstream of the nucleocapsid. The primers used are shown below. Vectorette primer sequences are proprietary and not known. These PCR amplifications were performed on a Stratagene Robocycler PCR machine and used Pfu Turbo DNA polymerase (Stratagene) to minimize nucleotide misincorporation.

First stage PCR:

Vectorette primer I (supplied in Genosys kit)

HRV-5 Nucleocapsid primer

5′- CTGAAATTGTTTCYGCCCTCACCT (SEQ ID NO: 64) (where Y is a C or a T)

Conditions: Initial denaturation at 95° C., 4 mins followed by 40 cycles of 95° C., 1 min 10 seas; 62° C., 1 min; 72° C., 6 mins.

One microliter of the first round products were transferred to the second stage.

Second stage PCR:

Vectorette nested primer II (supplied in Genosys kit) HRV-5 Nucleocapsid primer

5′-TGTGGCCCCATTTGAGGTGTTAG (SEQ ID NO: 66)

Conditions: Initial denaturation at 95° C. for 4 mins followed by 40 cycles of 95° C., 1 min 10 seas; 62° C., 1 min; 72° C., 6 mins.

When the second stage PCR products were analysed by agarose gel electrophoresis, a smear ranging from approximately 300 bp to 1500 bp was observed. These products were analyzed by Southern blotting and hybridized with a digoxygenin-labelled oligonucleotide probe specific for the HRV-5 nucleocapsid region (5′-GCTGTTGTCCATATACACCTGATC; (SEQ ID NO: 69) in order to identify any HRV-5 fragments present within this smear.

Hybridized probe was detected using reagents from Boehringer Mannheim (DIG detection kit). A band of approximately 800 bp was observed and the remainder of the PCR products were electrophoresed on an agarose gel and the appropriate region of the gel excised. DNA purified from this gel slice was subcloned into pBluescript (Stratagene) and plasmids containing HRV-5 sequences were identified and sequenced. The sequence obtained overlapped with the known nucleocapsid region of HRV-5 and extended to an EcoR I site 480 bp upstream of NC. This new fragment of the HRV-5 genome was designated Gagl. As expected, the region of HRV-5 nucleocapsid represented by the degenerate primer 8532 (nucleotides 1-26 in FIG. 1) was found to have a number of mismatches when compared to the genuine sequence of Gag1.

Subsequently, a primer based on the Gag1 sequence was used to clone a further region of the HRV-5 Gag gene using 1 μl of the second stage PCR products of the Cla I Vectorette-adapted DNA as template.

Primers used:

Vectorette sequencing primer (supplied in Genosys kit)

HRV-5 Gag primer (CA2R) 5′ CTGTACTATCTTAGTTAGGCTGTG

(SEQ ID NO: 70)

Conditions: Initial denaturation at 95° C. for 4 mins followed by 40 cycles of 94° C., 1 min 10 secs; 51° C., 1 min 10 secs; 72° C. 3 mins.

This PCR produced a clear band of approximately 1000 bp after analysis by agarose gel electrophoresis.

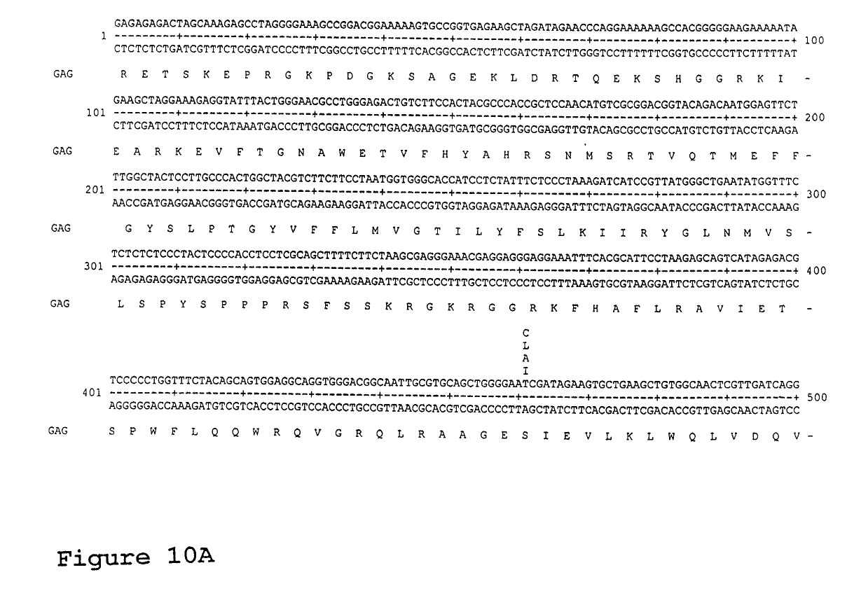

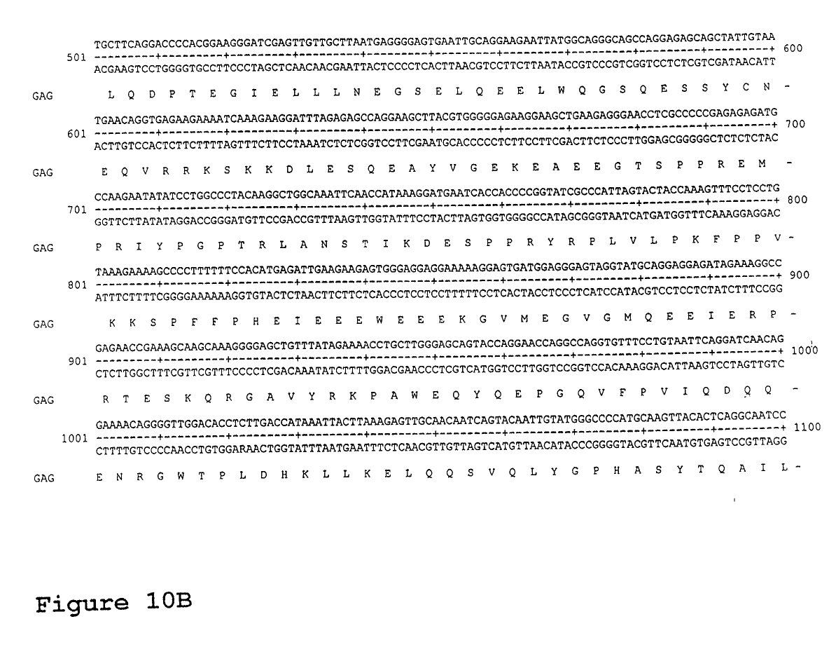

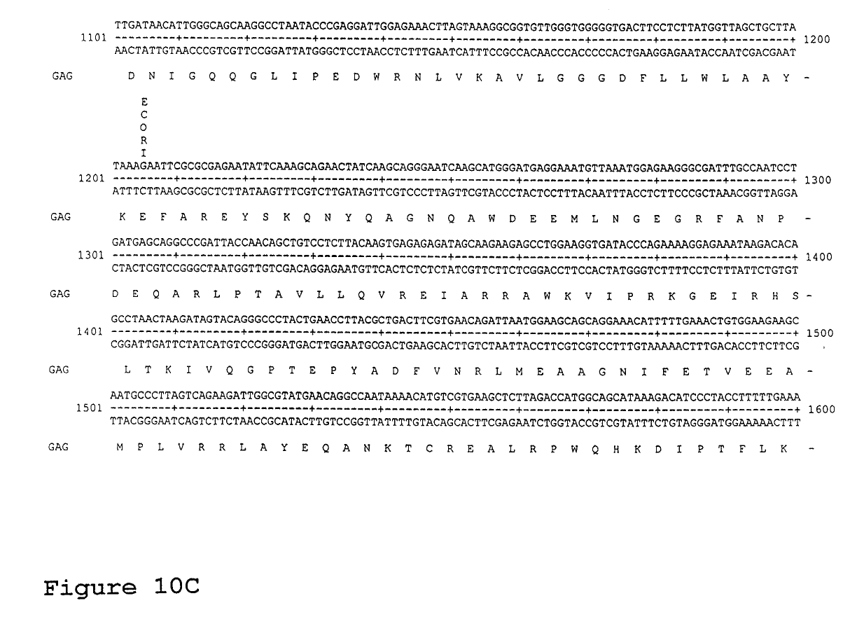

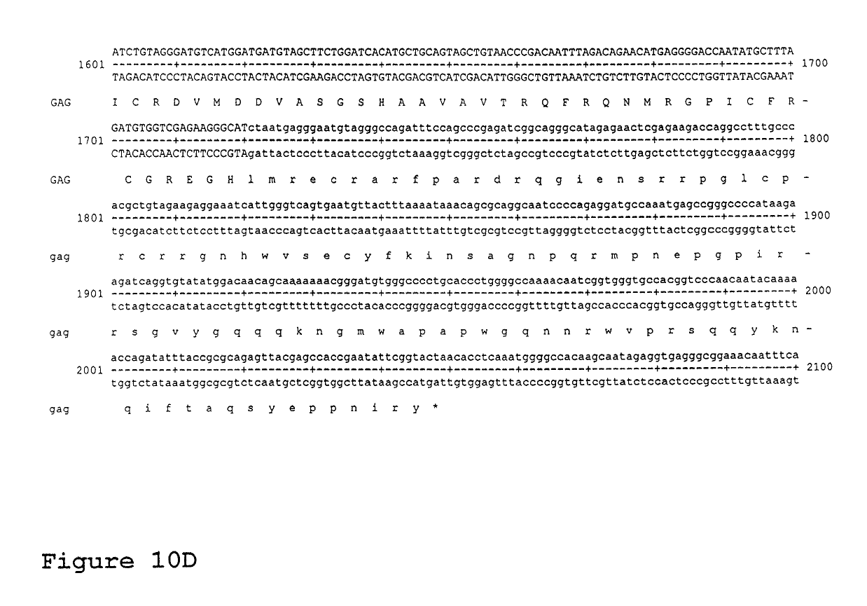

Following cloning and sequencing this product was found to extend the known HRV-5 gag sequence to a Cla I restriction site 1250 bp upstream of the original NC fragment. This second region (between the Cla I and Eco RI sites) was denoted Gag2. The composite sequence of HRV-5 Gag is shown in FIG. 10.

### EXAMPLE 9

Bacterial Expression of Recombinant HRV-5 Gag Protein

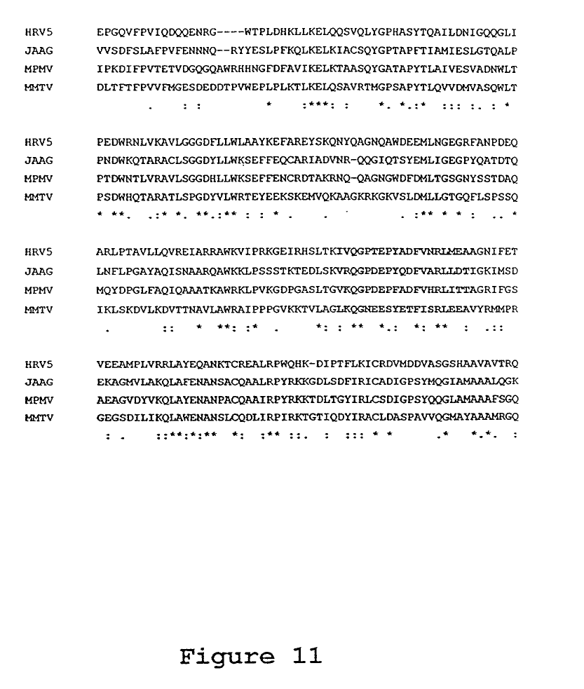

The major capsid protein of retroviruses (CA) is commonly used as a target antigen in immunological detection assays. In order to develop such an assay for HRV-5, a region of the gag protein of HRV-5 likely to represent CA was expressed in a bacterial expression system. The region of HRV-5 most likely to represent CA was identified by comparison with published sequences of other B and D-type retroviruses (FIG. 11). The appropriate DNA sequence was re-amplified from the cloned PCR fragments obtained previously, gel purified and blunt-end cloned into pBluescript using standard methods.

Primers used:

Forward (containing an Nco I site)

5′-AGAGACCATGGAACCAGGCCAGGTGTTTCCTG; (SEQ ID NO: 71)

Reverse (containing Xba I site)

5′- GAGATTCTAGAAATTGTCGGGTTACAGCTACTGC (SEQ ID NO: 72)

Conditions were 94° C. for 4 mins followed by 30 cycles of 94° C., 1 min 10 secs; 55° C., 1 min 10 secs; 72° C., 1 min 30 secs.

The resulting plasmids were sequenced to check that the PCR had not introduced errors into the Gag sequence and selected plasmids were then digested to completion with Xba I and partially digested with Nco I before subcloning the 700 bp HRV-5 CA fragment into the bacterial expression vector pTrcHis2B (Invitrogen) which had previously been digested with Nco I and Xba I. The resulting plasmid was designated pTrc-CA.

The pTrcHis2B expression vector was used because it allows the production of the desired protein fused to two epitope tags, namely a polyhistidine tag and a “myc” tag. The poly histidine (HIS-6) tag allows the purification of the desired protein using affinity chromatography on Nickel-agarose beads [Schmitt et al 1993, Mol. Biol. Rep., 18: 223-230]. The c-myc epitope tag allows the detection of the expressed protein in immunoblots using a monoclonal antibody specific for this epitope [Evan et al, 1985, Mol. Cell Biol. 5: 3610-3616].

A 2 ml culture of transformed bacteria containing plasmid pTrc-CA was grown overnight in Luria Bertani broth supplemented with 100 μg/ml ampicillin. This culture was then diluted 1 in 10 with fresh medium and grown for 1 hour at 37° C. with shaking. IPTG was then added to a final concentration of 1 mM in order to induce expression of the tagged HRV-5 CA protein and the culture grown for a further 90 mins. Extracts of the bacteria were then analysed by SDS-PAGE and production of the desired CA protein was confirmed by the presence of a 30 kDa protein following immunoblotting with the anti-myc monoclonal antibody, 9E10 [Evan et al, 1985, Mol. Cell Biol. 5: 3610-3616].

This protein was subsequently purified by metal chelate chromatography using a commercial kit (Xpress System, from Invitrogen) and was obtained substantially free of contaminating bacterial proteins. The recombinant HRV-5 CA protein is now ready for use as a target antigen in immunoblots, ELISAs and other serological assays for the detection of anti-HRV-5 antibodies in human sera. In addition the protein will be used to generate specific rabbit polyclonal and rat monoclonal antibodies as has already been achieved for the protease and reverse transcriptase proteins of HRV-5.

Cloning of HRV-5 Gag for Bacterial Expression

The HRV-5 Gag protein was PCR amplified and subcloned into the bacterial expression vector pTrcHIS2B (Invitrogen). This vector provides C-terminal c-myc and polyhistidine tags to facilitate detection and purification of the expressed protein.

Primers used:

Forward; SEQ ID NO: 105:
ATGGAACGACCATGGAGTTCTTTGGCTACTCTTTG;
Reverse: SEQ ID NO:106
GAGATCTAGATTAGTACCGAATATTCGGTGACTCGTA

HRV-5 Gag was amplified from HRV-5 plasmid DNA in a 50 microliter reaction volume containing 10 pmol of each primer, using pfuTurbo DNA polymerase (Stratagene) as recommended. Conditions were 30 cycles of 94° C., 45 secs; 55° C., 45 secs; 72° C., 2 mins, with an initial denaturation at 94° C. for 4 minutes. The PCR product was gel-purified, digested with NcoI and XbaI (sites contained in the primers) and ligated into digested pTrcHis2B to generate plasmid pTrcGag. The plasmid was sequenced to confirm the construct was as desired.

For Gag expression in E. coli, BL21 CodonPlus cells (Stratagene) were transformed with pTrcGag using standard methods. Protein expression was induced in selected transformants by inoculating a 2 ml of LB-broth containing 100 μg/ml ampicillin and 150 μg/ml chloramphenicol with a single colony and culturing overnight at 37° C. with shaking at 300 rpm. The culture was diluted 1 in 10 with fresh medium/antibiotics and grown for 1 hour at 37° C. with shaking. Protein expression was induced by addition of IPTG to a final concentration of lmM and growing for a further 3 hours. Protein was detected by western blotting using an anti-myc monoclonal antibody.

The HRV-5 Gag protein was purified by metal chelate affinity chromatography from 100 ml cultures (grown and induced under similar conditions as aboveand scaled up) using procedures recommended by the manufacturer. Bacterial pellets were resuspended in 5 ml/g wet weight of extraction buffer (6 M guanidine hydrochloride, 100 mM NaH₂PO₄, 10 mM Tris-HCl pH 8.0) and lysed by incubation on ice for 15 mins followed by brief sonication on ice (2×30 second bursts with a 30 second gap; MSE soniprobe). The lysate was then centrifuged at 10,000 rpm (in Beckman JA-20) for 10 minutes at 4° C. and the supernatant removed. Triton X-100 (final concentration 1%), β-mercaptoethanol (10 mM) and imidazole (10 mM) were then added followed by 750 μl of a 50% slurry of nitrolotriacetic acid-Ni²⁺-Sepharose (NTA-Ni²⁺) beads (Qiagen; prewashed three times in extraction buffer). The samples were mixed for 1 hour at room temperature.

After binding of proteins, the NTA-Ni²⁺ beads were washed with 50 bed volumes of extraction buffer pH 8.0, 50 bed volumes of wash buffer (6 M urea, 100 mM NaH₂PO₄, 10 mM Tris-HCl pH 6.3) and 50 bed volumes of wash buffer containing 25 mM imidazole (pH 6.3). Proteins were then eluted from the NTA-Ni²⁺ beads with 2 bed volumes of extraction buffer containing 100 mM imidazole pH 6.3 and 2 bed volumes of extraction buffer with 250 mM imidazole pH 6.3. Fractions were taken at various stages of washing for analysis by SDS-PAGE, Coomassie staining and immunoblotting.

### EXAMPLE 10

Cloning of an Additional Fragment of HRV-5 Gag

In addition to Vectorette PCR, other DNA-walking methods were used to extend the HRV-5 sequence. A further 260 bp was cloned using a procedure based on 5′ RACE (5′ amplification of cDNA ends, Frohman et al., 1988, Proc Nat Acad Sci USA. 85: 8998-9002) which was adapted for use on genomic DNA. The adaptations were designed to enrich the target DNA with HRV-5 sequences and to prepare single stranded DNA which may serve as a template for the tailing step of the RACE reaction.

Three micrograms of DNA from normal blood were added to a PCR reaction containing a single primer specific for HRV-5.

Primer:

HRV-5 Gag primer (CA2R1)

5′-(biotin)-GCTTCCTGGCTCTCTAAATCCTTC (SEQ ID NO: 73)

Conditions were an initial denaturation at 94° C. for 4 mins followed by 40 cycles of 94° C., 1 min; 62° C., 1 min 10 secs; 72° C., 3 mins.

The HRV-5 specific primer used in this reaction was modified in that it was synthesised with a biotin molecule at its 5′ terminus. The purpose of this single primer PCR was to generate single stranded DNA molecules extending from the known region of HRV-5 Gag into the upstream flanking sequence. These single stranded DNA molecules were purified using streptavidin coated magnetic beads by utilising the 5′ biotin modification of the primer used in the PCR. This purification was performed using the KilobaseBINDER kit (Dynal, Sweden) as recommended.

The selected DNA fragments were then further modified by the addition of a “tail” of deoxyadenosine nucleotides to the 3′ end of the purified DNA. This was achieved using terminal transferase and DATP and utilised reagents in the 3′ and 5′ RACE kit (Boehringer) essentially as recommended.

The selected, tailed single stranded DNA molecules were then subjected to PCR amplification using HRV-5 specific primers (from the known Gag2 region) and primers designed to the synthetic oligo dA tail (provided in the RACE kit).

First stage primers:

HRV-5 Gag primer (CA2R2) 5′-CTCACCGGTTCATTACAATAGCTGC (SEQ ID NO: 74)

Anchor tailed primer:

5′-GACCACGCGTATCGATGTCGACTTTTTTTTTTTTTTTTV (Where V is a C a G or an A; SEQ ID NO: 75).

Conditions were an initial denaturation at 94° C. for 4 mins followed by 40 cycles of 94° C., 1 min 10 secs; 55° C., 1 min 10 secs; 72° C., 3 mins 30 secs. One microliter of first round products were transferred to the second stage.

Second stage primers:

HRV-5 Gag primer (CA2R3) 5′-GCTGCCCTGCCATAATTCTTCCTG (SEQ ID NO: 76)

Anchor primer: 5′-GACCACGCGTATCGATGTCGAC (SEQ ID NO: 77)

Conditions were an initial denaturation at 94° C. for 4 mins followed by 40 cycles of 94° C., 1 min 10 seas; 55° C., 1 min 10 seas; 72° C., 3 mins 30 secs.

Cloning and sequencing of the second stage PCR products established the sequence of a further region of HRV-5 upstream of Gag2. This region was denoted Gag3.

This modified RACE procedure was subsequently used to clone 2 additional fragments of the HRV-5 gag gene, denoted Gag4 and Gag5. The Gag4 fragment was amplified from 1 mg of DNA from the colon tissue of a patient with ulcerative colitis.

Single primer PCR:

HRV-5 Gag Primer (CA3R1) 5′-(biotin)-TCCCACCTGCCTCCACTGCTGTAG (SEQ ID NO: 78)

Conditions were an initial denaturation at 94° C. for 4 mins followed by 40 cycles of 94° C., 1 min; 62° C., 1 min 10 seas; 72° C., 3 mins. Single stranded extension products were purified using streptavidin coated magnetic beads as for the Gag3 fragment. A polynucleotide tail was then added as for the Gag3 fragment except that in this case a deoxygaunosine tail was added instead of polyadenosine. The selected, tailed single stranded DNA molecules were then subjected to PCR amplification using HRV-5 specific primers (from the known Gag3 region) and primers designed to the synthetic oligo dG tail.

First stage primers:

HRV5 Gag primer (CA3R2) 5′-ACCAGGGGGACGTCTCTATGACTG (SEQ ID NO: 79)

Anchor tailed primer:

5′- GACCACGCGTATCGATGTCGACCCCCCCCCCCCCCCCD

(where D is an A, a G or a T; (SEQ ID NO: 80).

Conditions were an initial denaturation at 94° C. for 4 mins followed by 40 cycles of 94° C., 1 min 10 secs; 55° C., 1 min 10 secs; 72° C., 3 mins 30 secs. One microliter of first round products were transferred to the second stage.

Second stage primers:

HRV-5 Gag primer (CA3R3) 5′-CTTAGGAATGCGTGAAATTTCCTC (SEQ ID NO: 81)

Anchor primer: 5′-GACCACGCGTATCGATGTCGAC (SEQ ID NO: 77)

Conditions were an initial denaturation at 94° C. for 4 mins followed by 40 cycles of 94° C., 1 min 10 secs; 55° C., 1 min 10 secs; 72° C., 3 mins 30 secs.

Cloning and sequencing of the second stage PCR products established the sequence of a further 45 bp of HRV-5 upstream of Gag3. This region was denoted Gag4. This procedure was then repeated to clone the Gag5 fragment.

Single primer for extension reaction (from HRV5 Gag):

5′-(biotin)-GCATTCAGCCCATAACGGATGATC (SEQ ID NO: 82)

Conditions were an initial denaturation at 94° C. for 4 mins followed by 40 cycles of 94° C., 1 min; 61° C., 1 min; 72° C., 2 mins. Single stranded extension products were purified using streptavidin coated magnetic beads as for the Gag3 fragment. A polydeoxygaunosine tail was then added as for the Gag4 fragment. The selected, tailed single stranded DNA molecules were then subjected to PCR amplification using HRV-5 specific primers (from the known Gag3 region) and primers designed to the synthetic oligo dG tail.

First stage primers:

HRV5 Gag primer (CA4R2) 5′-AAGATGTAGCCAGTGGGCAAGGAG (SEQ ID NO: 83)

Anchor tailed primer:

5′-GACCACGCGTATCGATGTCGACCCCCCCCCCCCCCCCD

(where D is an A, a G or a T; SEQ ID NO: 80). Conditions were an initial denaturation at 95° C. for 4z mins followed by 40 cycles of 94° C., 1 min 10 secs; 55° C., 1 min 10 secs; 72° C., 2 mins 30 secs. One microliter of first round products were transferred to the second stage.

Second stage primers:

HRV-5 Gag primer (CA4R3) 5′-GTAGCCAAAGAACTCCATTGTCTG (SEQ ID NO:84)

Anchor primer: 5′-GACCACGCGTATCGATGTCGAC (SEQ ID NO: 77)

Conditions were an initial denaturation at 95° C. for 4 mins followed by 40 cycles of 94° C., 1 min 10 secs; 55° C., 1 min 10 secs; 72° C., 2 mins 30 secs.

Cloning and sequencing of the second stage PCR products established the sequence of a further 160 bp of HRV-5 upstream of Gag4. This region was denoted Gag5. In total the 3 extension products obtained using the modified RACE procedure yielded 458 bp of sequence information upstream of the ClaI site of the Vectorette PCR products. The composite sequence of HRV-5 Gag is shown in FIG. 10.

The composite sequence of HRV-5 Gag is shown in FIG. 10.

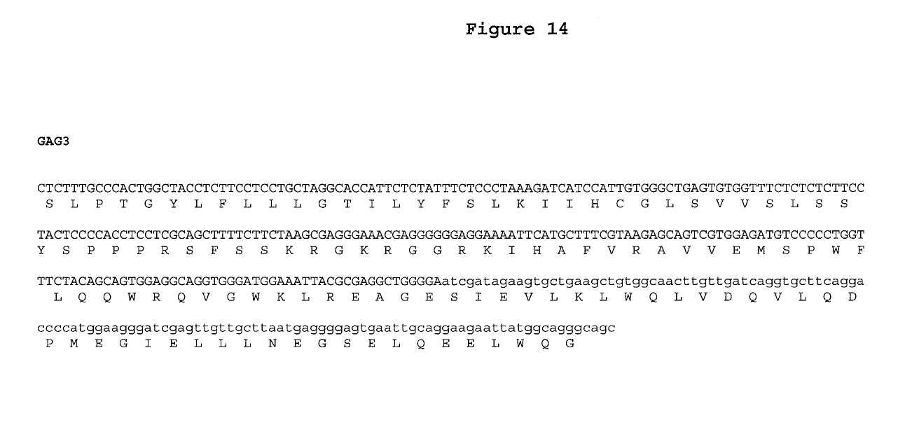

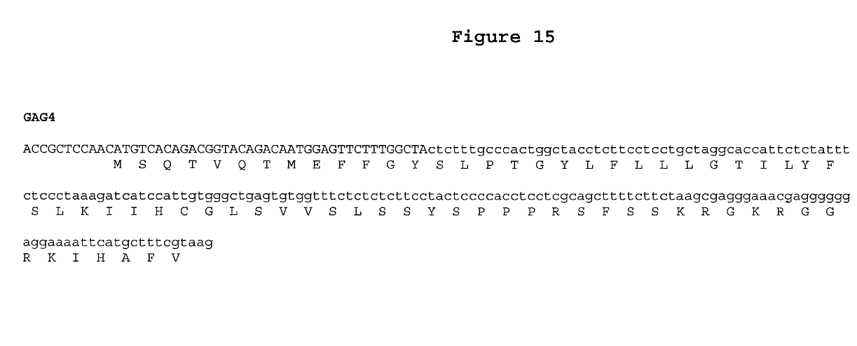

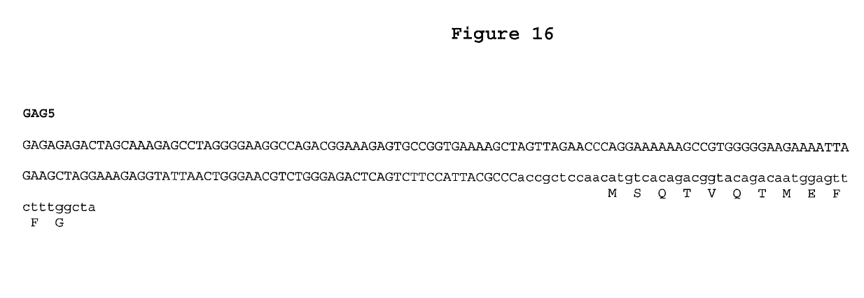

Extended Gag3 sequences are shown in FIG. 14; nucleic acid sequence, SEQ ID NO: 92, amino acid sequence, SEQ ID NO: 93. Extended Gag4 sequences are shown in FIG. 15; nucleic acid sequence, SEQ ID NO: 94; amino acid sequence, SEQ ID NO: 95. Gag5 sequences are shown in FIG. 16, SEQ ID NO:SEQ ID NO: 96, amino acid sequence, SEQ ID NO: 97.

In total, using the RACE PCR we successfully cloned 458 bp of the HRV-5 genome comprising the 5′ terminus of the gag gene and a large region of the 5′ untranslated leader sequence. The final fragment of HRV-5 leader sequence and a part of the 5′ long terminal repeat was cloned using Vectorette PCR.

The Vectorette PCR system involves restriction enzyme digestion of the target DNA and subsequent ligation of double stranded oligonucleotide linkers (‘Vectorettes’) to the cut DNA ends. In practice, the target DNA is digested (separately) with a number of different enzymes in order to maximize the probability that one of these restriction sites is within a suitable range for PCR amplification. In addition, the oligonucleotide linker is designed in such a way that non-specific amplifications are minimized (Arnold and Hodgson, 1991, PCR Methods Appl. 1: 39-42).

For cloning the final fragment of the HRV-5 leader region, Vectorette PCR was performed using a kit obtained from Genosys Biotechnologies (UK). Five micrograms of DNA from the blood of an apparently normal individual was digested with the restriction enzyme Nsp I and blunt-ended Vectorette linkers were ligated on to the cut DNA.

Three rounds of digestion and ligation were performed to minimize concatamer formation as recommended in the manufacturer's protocol. Nested PCR was then performed on aliquots of the ligation products using HRV-5 specific primers in conjunction with primers derived from the linker sequence. This experiment resulted in the amplification of sequences upstream of the Gag5 region The primers used are shown below. Vectorette primer sequences are proprietary and not known. These PCR amplifications were performed on a Stratagene Robocycler PCR machine and used Pfu Turbo DNA polymerase (Stratagene) to minimize nucleotide misincorporation.

First stage primers:

HRV-5 specific primer (CA4R3)

5′- GTAGCCAAAGAACTCCATTGTCTG; SEQ ID NO: 84)

Vectorette primer (Genosys)

Amplification conditions were 40 cycles of 95° C., 1 minute 10 seconds; 55° C., 1 minute 10 seconds; 72° C., 3 minutes with an initial denaturation at 95° C. for 4 minutes. 1 μl of the first stage products were transferred to the second stage PCR.

Second stage primers:

HRV-5 specific primer (GAG6R2)

5′-TTGGAGCGGTGGGCGTARTGGAAGG; SEQ ID NO: 107Vectorette nested primer (Genosys)

Where R is an A or a G.

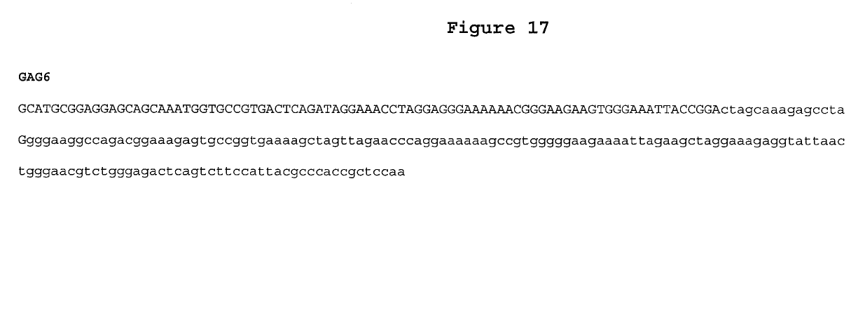

Conditions were 40 cycles of 95° C., 1 minute 10 seconds; 60° C., 1 minute 10 seconds; 72° C., 3 minutes with an initial denaturation at 95° C. for 4 minutes). Analysis of the products of this PCR identified an 85 bp region upstream of Gag5 which extended the known sequence of HRV-5 into the 5′ long terminal repeat. This fragment was designated Gag6. See FIG. 17; (SEQ ID NO: 98).

### EXAMPLE 11

Cloning an additional fragment of HRV-5 Integrase

The degenerate primer and RACE PCR cloning methods were combined to clone an additional 260 bp fragment of the HRV-5 integrase gene. 1 μg of DNA from colon tissue of a patient with ulcerative colitis was added to a RACE primer extension reaction as described in Example 10 for the Gag3 fragment.

Primer for single primer PCR (from HRV-5 pol gene):

Primer (INF6bio) 5′-(biotin)-GTTGCCATAGTTCCAAAGATTCCTG; (SEQ ID NO: 85)

Conditions were an initial denaturation at 95° C. for 4 mins followed by 40 cycles of 94° C., 1 min; 61° C., 1 min; 72° C., 2 mins. Single stranded extension products were purified using streptavidin coated magnetic beads as for the Gag3 fragment. A polydeoxyadenosine tail was then added as for the Gag3 fragment. The selected, tailed single stranded DNA molecules were then subjected to PCR amplification using HRV-5 specific primers (from the known Gag3 region) and primers designed to the synthetic oligo dA tail.

First stage PCR

HRV5 Pol primer (INF6) 5′-GTTGCCATAGTTCCAAAGATTCCTG (SEQ ID NO: 85)

Anchor tailed primer:

5′- GACCACGCGTATCGATGTCGACTTTTTTTTTTTTTTTTV (SEQ ID NO: 75)

(Where V is a C a G or an A).

Conditions were an initial denaturation at 95° C. for 4 mins followed by 40 cycles of 94° C., 1 min 10 secs; 55° C., 1 min 10 secs; 72° C., 2 mins. One microliter of first round products were transferred to the second stage.

Second stage primers:

HRV-5 Pol primer (INF7) 5′-GAGCCAATCCCCGTGGCCTTAAAC (SEQ ID NO: 86)

Degenerate retrovirus integrase primer (IN-GIPl): 5′-YTGKCCYTGKGGATTRTARGG (SEQ ID NO: 86)

(Where Y is a C or a T; K is a G or a T and R is an A or a G).

Conditions were an initial denaturation at 94° C. for 4 mins followed by 35 cycles of 94° C., 1 min 10 secs; 50° C., 1 min 30 secs; 72° C., 1 min 20 secs.

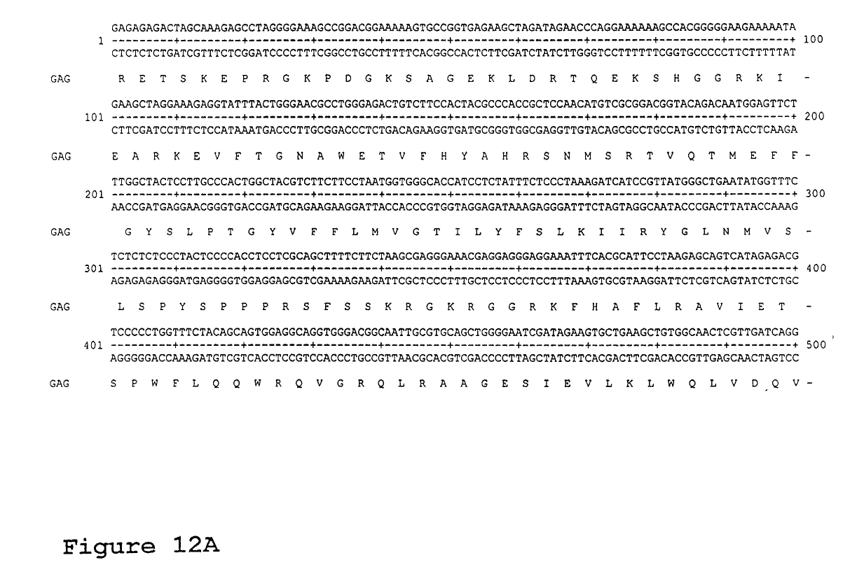

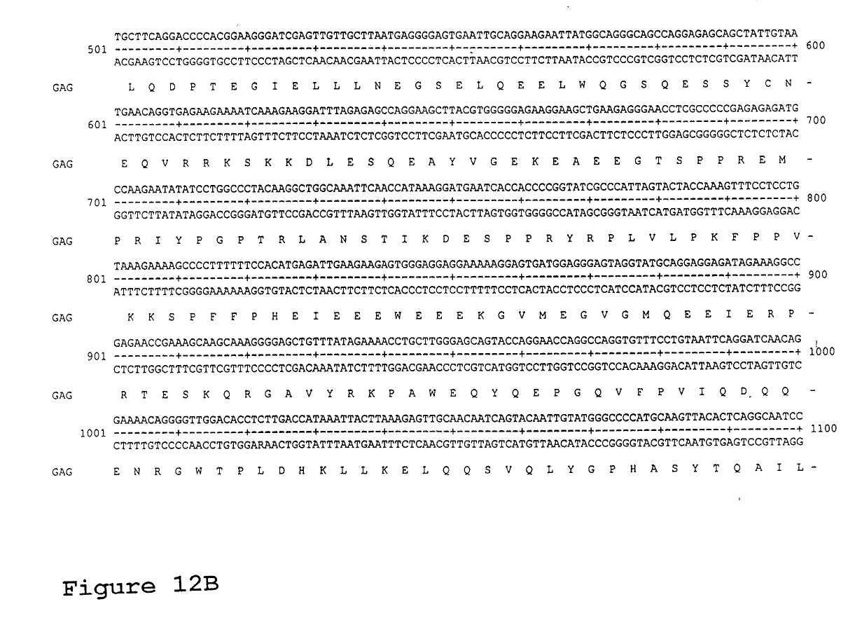

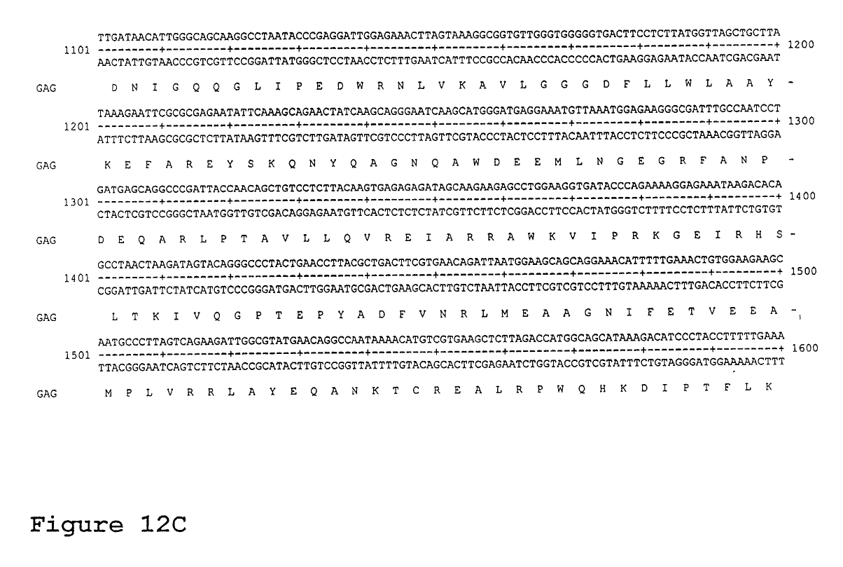

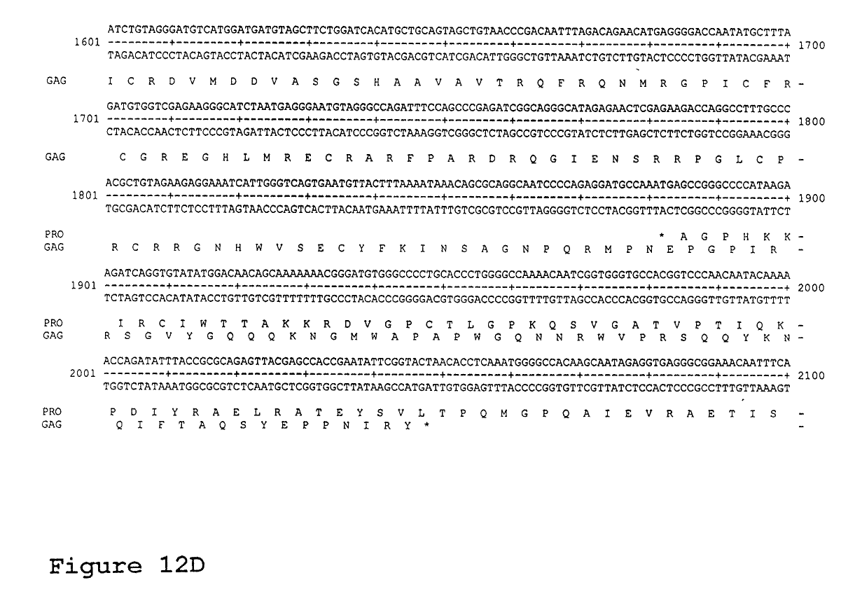

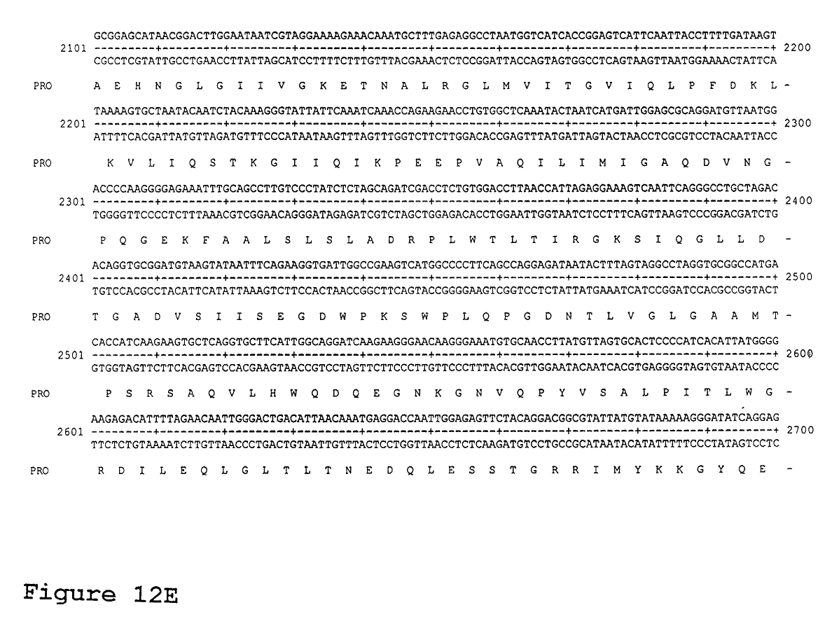

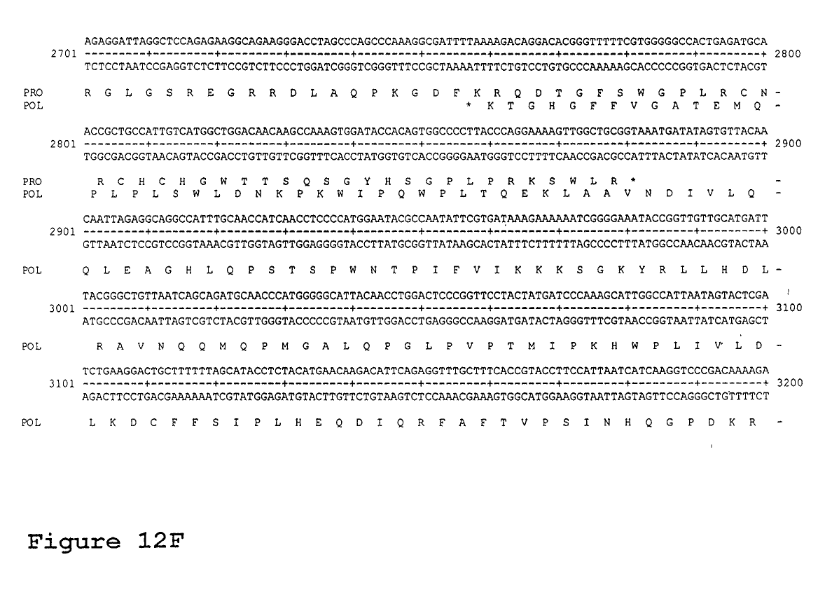

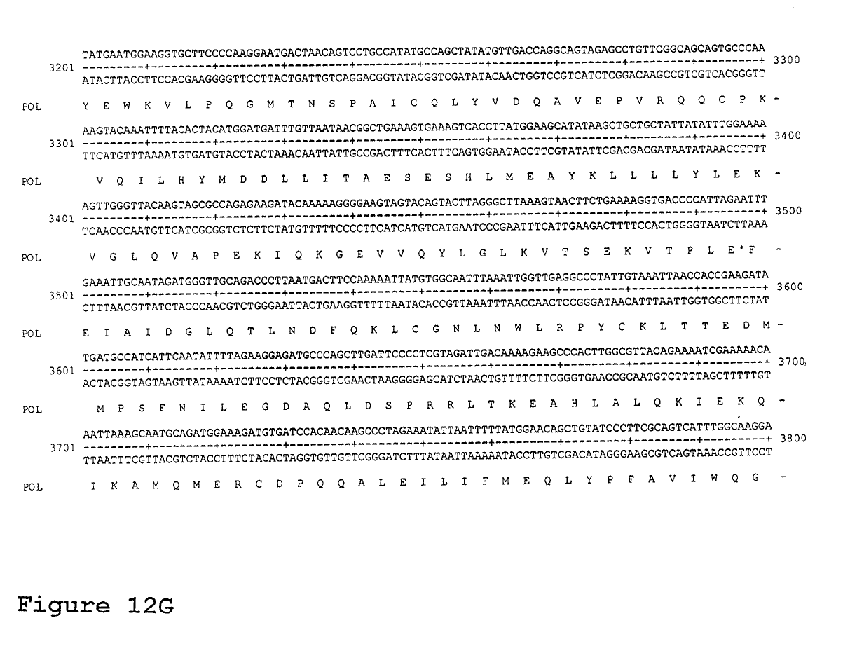

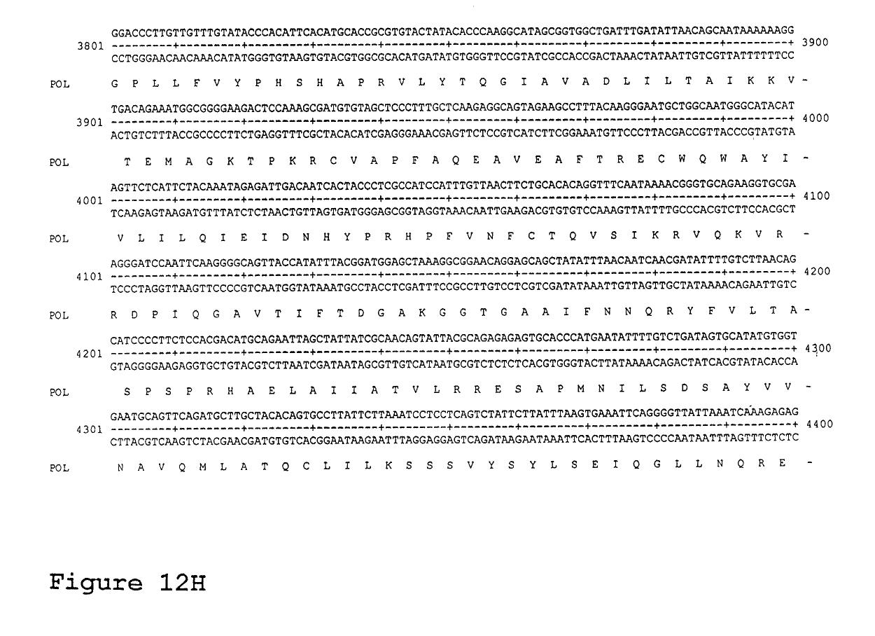

Cloning and sequencing of the second stage PCR products established the sequence of a further 260 bp of the HRV-5 pol gene. This region was denoted IN2. This region represents nucleotides 4648 to 4693 of the sequence presented in FIG. 12. Note that nt 4941 to 4961 are derived from the degenerate primer IN-GIP1 and so may not represent the genuine sequence of HRV5 in this region.

### EXAMPLE 12

Accession Number

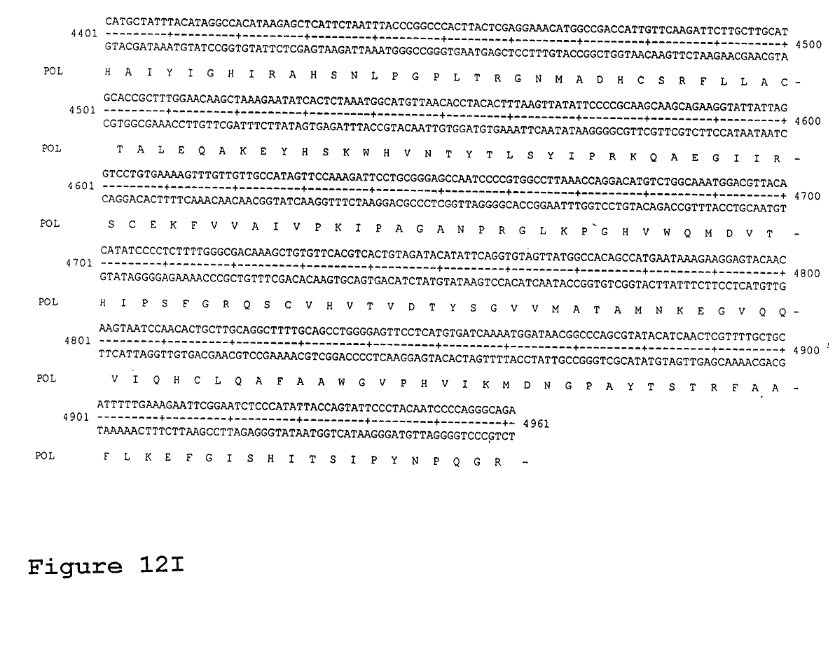

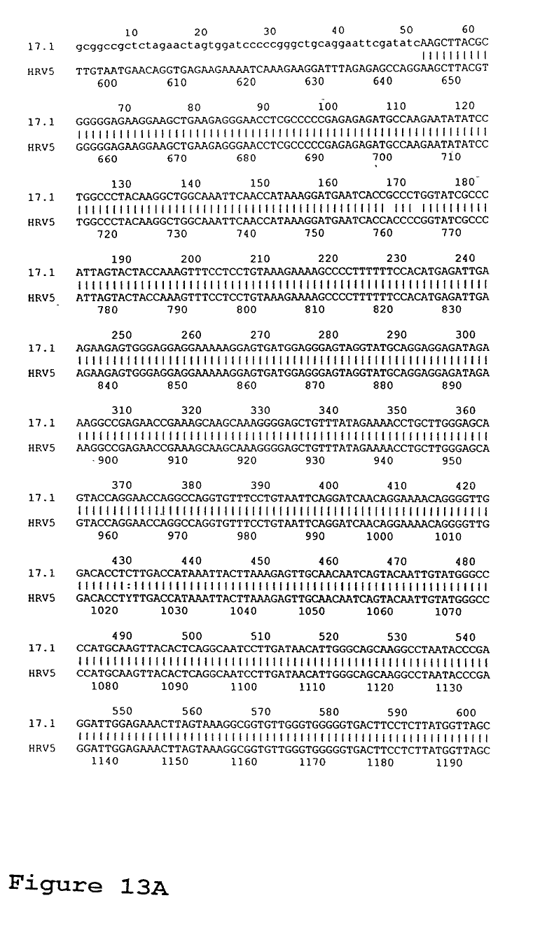

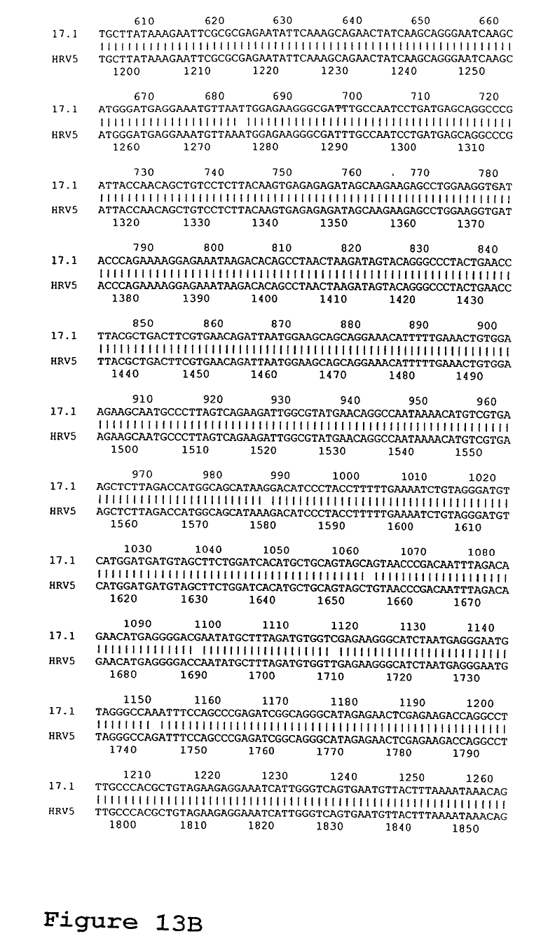

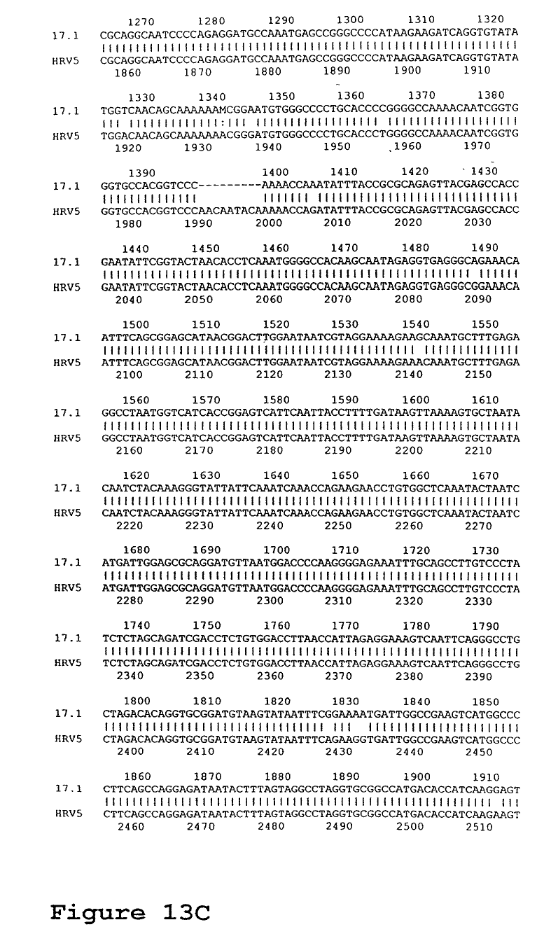

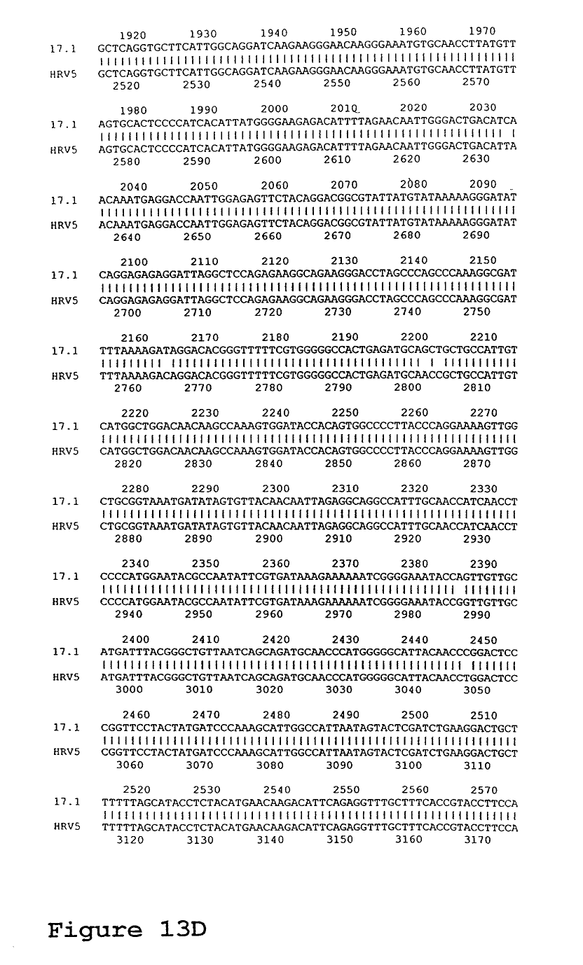

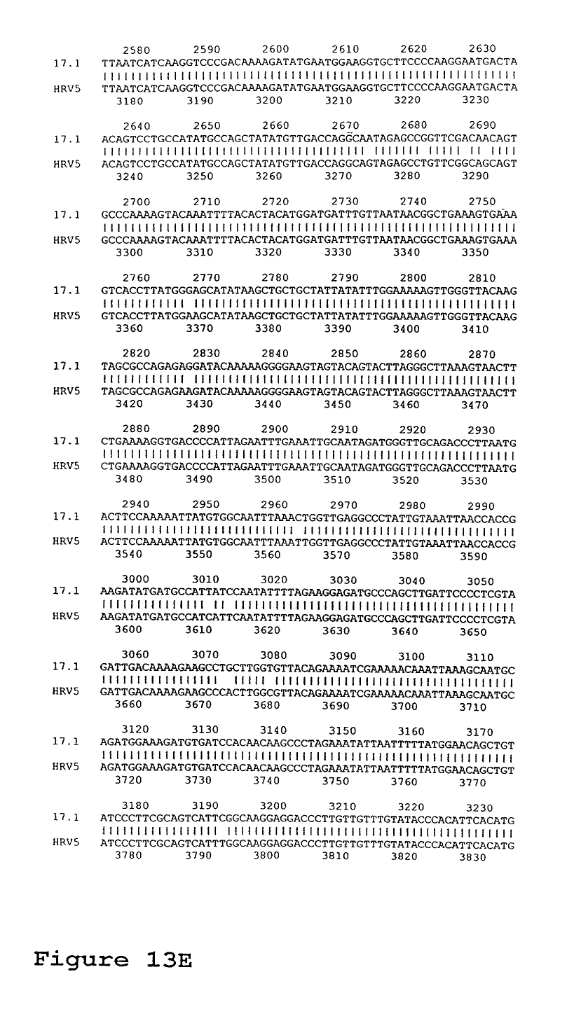

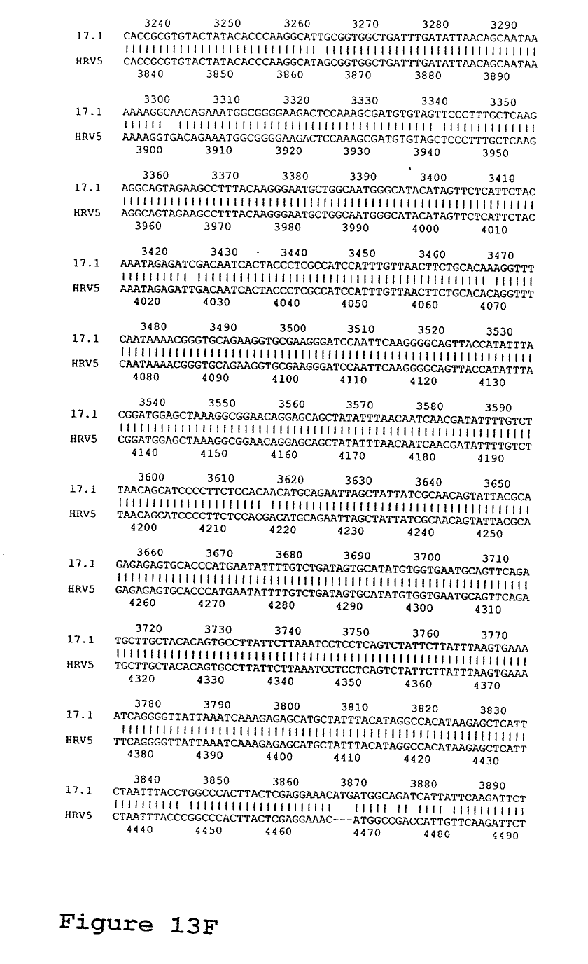

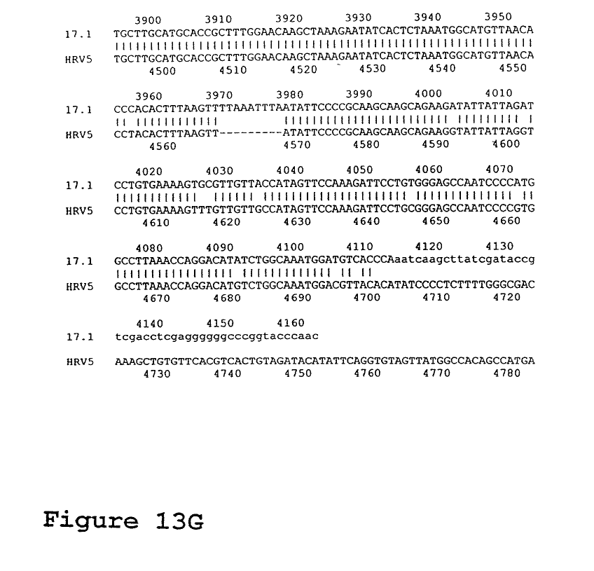

A plasmid (pHRV5gagpol 17.1) containing HRV-5 gag, pro and pol genes was deposited with the European Collection of Cell Cultures (ECACC) on 19 March 1999 under the Accession number 99031901. This plasmid was constructed from the various PCR amplified fragments of HRV-5. Since the sequences shown in FIG. 12 represents the consensus sequence of the various PCR fragments, the plasmid pHRV5gagpol 17.1 has a number of nucleotide differences from this consensus sequence. These differences are shown in FIG. 13. The applicants give their unreserved and irrevocable consent to the materials being made available to the public in accordacne with appropriate national laws governing the deposit of these materials, such as Rules 28 and 28a EPC. The expert solution under Rule 28(4) EPC is also hereby requested.

### Example 13

Bacterial Expression and Antibody Production.

In order to develop antisera and monoclonal antibodies (Mabs) for the detection of viral proteins in primary tissue and in culture, fragments of the potential gag, pro and pol proteins of HRV-5 have been expressed using the bacterial expression vector pTrc99A (Pharmacia) in the M15 [pREP4] (Qiagen) bacterial host strain. This has been accomplished using PCR amplified regions of the appropriate HRV-5 genes. In addition to gene-specific nucleotides the 5′ PCR primers also contained nucleotides encoding 6 consecutive histidine residues (His₆-tag) to facilitate purification of the proteins by means of a Ni²⁺-containing resin marketed by Qiagen (Ni²⁺-NTA resin). The 3′ primers also included nucleotides encoding a 10 amino-acid epitope from the human c-myc gene to enable detection of the proteins by western blotting with a monoclonal anti-c-myc antibody (9E10) specific to this epitope (Evan et al. 1985, Mol. Cell Biol. 5: 3610-3616). In addition to the His₆ and c-myc sequence tags, the PCR primers also contained restriction sites to enable cloning into the pTrc99A vector.

Proteins were purified using Ni²⁺-NTA resin (Qiagen). Overnight cultures of bacteria carrying the subcloned fragments of HRV-5 were grown in Luria-Bertani broth supplemented with ampicillin (100 μg/ml) and kanamycin (25 μg/ml). The next day these were diluted 1:10 into fresh antibiotic-containing broth and grown at 30° C. for one hour (optical density at 600 nm approximately 0.6). Expression of the proteins was then induced by addition of iso-propyl-thio-galactoside (IPTG; 1 mM) and culture continued for a further 90 minutes (OD₆₀₀=0.9). Cells were pelleted by centrifugation and resuspended in NTA-purification buffer pH 8.0 (8M urea, 100 mM NaH₂PO₃, 10 mM TRIS-Cl). Cells are then lysed by three cycles of freeze-thawing followed by brief sonication. Clarified lysates are then incubated with Ni²⁺-NTA resin for four hours at 4° C. and then poured into a chromatography column support (Bio-Rad). Contaminating proteins are washed off with NTA-purification buffer pH 6.3 containing 25 mM imidazole. Finally the purified proteins are eluted from the resin with NTA purification buffer pH 6.3 containing 250 mM imidazole.

Fragments of HRV-5 proteins have also been expressed in E.Coli and purified using the glutathione-S-transferase (GST) system (Pharmacia). These proteins were used to raise polyclonal antisera in rabbits. These antisera are used as control antibodies for the ELISAs (discussed below).

The identity of the purified proteins can be confirmed by western blotting using the 9E10 Mab specific for the c-myc epitope tag. The proteins can then be used to raise rabbit polyclonal antisera in a known manner, preferably with the use of affinity purification to improve the specificity of the sera.

Antibody Production

Rat monoclonal antibodies specific for the HRV-5 proteins may be produced. CBH/Cbi rats may be immunized 4 times at 21 day intervals with 100 μg of either the PR or RT protein. The third immunization is preferably given via the intra-peritoneum, the other three immunizations via Peyer's patches. The immunogens may be emulsified in complete Freunds adjuvant (Difco Labs) prior to the first inoculations and in incomplete Freunds for subsequent immunizations.

Three days after the last immunization, mesenteric lymph node cells may be fused with rat myeloma Y3-Ag 1,2,3, [Dean et al., 1986, Methods in Enzymology, Vol 121, pp 52-59]. Supernatants from the resulting hybridomas are then screened for antibodies to the immunizing antigen by ELISA and by immunoblot.

ELISA plates are prepared by coating them with immunizing antigen at a concentration of lug/ml in PBS and incubating overnight at 4° C. Hybridoma supernatants can then be screened for binding to the immunizing antigen. After incubation for about 1 hour at room temperature, the plates are washed 3 times in wash buffer (PBS, 0.1% BSA, 0.05% Tween-20). Bound rat antibody may be detected using goat anti-rat immunoglobulin conjugated to horseradish peroxidase (Seralab) and incubated at room temperature for about 1 hour. Plates may be washed 3times in wash buffer and bound antibody detected by TMB (Sigma) to produce a soluble blue end product developed over 20 minutes. Acidification with 0.5 M H₂SO₄ stopping solution produces a yellow colour which may be read using a microplate autoreader at 450 nm.

Candidate hybridoma supernatants identified by ELISA may be used to probe immunoblots of the immunizing proteins. The supernatants can be used at dilutions of 1:200-1:25 and detected with goat anti-rat immunoglobulin-horseradish peroxidase conjugate (Harlin SeraLab, diluted 1:2000) and enhanced chemiluminescence (ECL, with reagents supplied by Amersham).

Mouse Mabs may also be prepared by methods which are conventional in the art.

ELISAs and Immunofluorescence

ELISAs for the detection of antibodies to HRV-5 proteins may be developed. An indirect ELISA and a capture ELISA system can be produced.

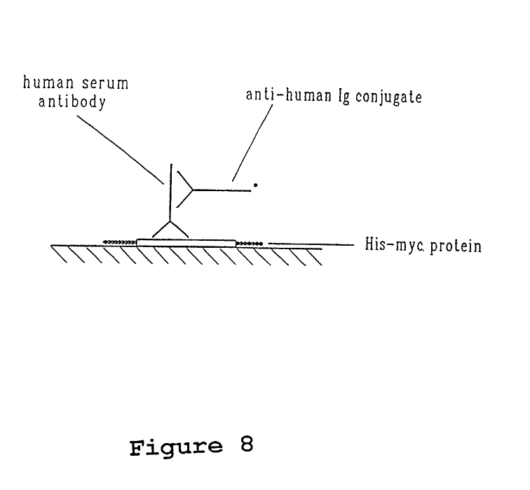

Indirect ELISA (FIG. 8)

ELISA plates are coated with recombinant HRV-5 proteins or synthetic peptides derived from HRV-5 proteins (50 μl; 5 μg/ml) and incubated at 4° C. overnight. The plates are then washed 3 times with PBS (100 μl per well), blocked with PBS/2% casein (100 ml/well) for 1hour at 37° C. and washed again 3 times with PBS. Test sera and standard control sera (50 μl; prepared in PBS/0.5% casein) are incubated on the plates at various dilutions for 1 hour at 37° C. and the plates washed 3 times in PBS. An anti-human alkaline phosphatase conjugate (Sigma 1:1000 dilution) is then incubated on the plates for 1 hour, 37° C. and washed 4 times in PBS, once in PBS/0.1% Tween 20 and then once in PBS (100 μl per well for each wash). Conjugates to human IgG and IgM can be used which may allow the distinction between early HRV-5 infection (IgM antibodies) and established HRV-5 infection (IgG). For control wells using polyclonal rabbit antisera or rat monoclonal antibodies, goat anti-rabbit IgG or goat anti-rat IgG conjugates are used respectively. Alkaline phosphatase substrate (Sigma-104; 50 μl/well) is then added to yield a yellow end product read at 405 nm with a microplate autoreader (BioTek Instruments).

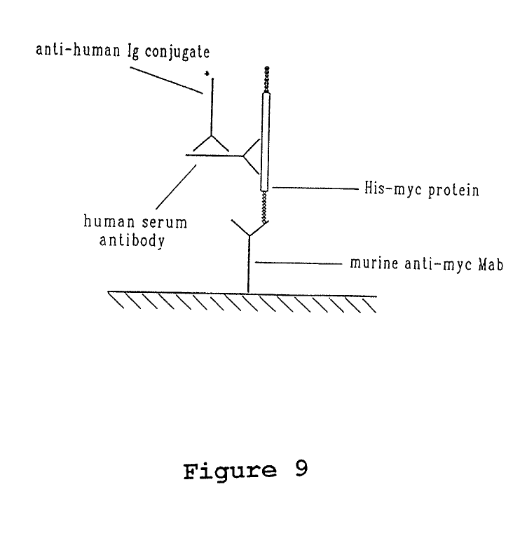

Capture ELISA (FIG. 9)

ELISA plates are coated with an anti-c-myc monoclonal antibody (9E10, Evan et al, 1985, Mol. Cell. Biol. 5: 3610-3616) (5 μg/ml; 50 μl/well) overnight at 4° C. Plates are then washed 3 times in PBS, blocked with PBS/2% casein (100 μl/well) for 1 hour at 37° C. and washed with PBS as before. Recombinant HRV-5 protein is then bound to the plates (as above for indirect ELISA) and the plates are washed 3 times with PBS (100 μl/well). Test sera are then incubated on the plates and detected using the alkaline phosphatase conjugate as described above for the indirect ELISA. The use of a capture ELISA may increase specificity of the ELISA since minor bacterial contaminants in the recombinant protein preparations will not bind to the 9E10-coated plates.

All ELISA results may be confirmed by immunoblotting.

Immunofluorescence

The anti-HRV-5 monoclonal antibodies may be used to examine human tissue sections by indirect immunofluorescence. Tissue sections (6 μm thick) were cut in OCT compound (Miles Diagnostics), fixed in 1:1 acetone/methanol at −20° C. and air-dried. The sections are then incubated with 50 μl of diluted test antibody for 30 mins at room temperature and washed twice in PBS (5 mins) and once in water. Bound antibodies are then detected using an anti-rat IgG fluorescein isothiocyanate conjugated antibody (Sigma F1763; 50 μl) for 30 mins at room temperature. The slides are then washed twice with PBS (5 mins) and once in water before mounting in glycerol with 2.5% (w/v) 1,4 diazobicyclo-2.2.2. octane and viewing under ultraviolet light.

As mentioned above the HRV-5 Gag polyprotein has been expressed and purified as a polyhistidine tagged protein in E. coli. This protein was then used as the target antigen in western blots with human sera. In these experiments we have identified 2 sera from patients with systemic lupus erythematosus which reacted with the HRV-5 Gag protein.

Epitone Mapping

In order to identify specific epitopes of HRV-5 Gag that are reactive with lupus sera, we generated a series of overlapping peptides (15 mers) derived from the N-terminus of Gag. This region corresponds to the matrix (MA) domain of retroviral Gag polyproteins. These peptides were synthesised on a cellulose membrane using the SPOTS kit (Sigma-Genosys) as recommended and exposed to the positive sera identified by western blot. 2peptides showed strong reactivity with these sera. We then prepared large quantities of these peptides for use in ELISAs. The peptide sequences were SFSSKRGKRGGRKIHC; SEQ ID NO: 108 and PWFLQQWRQVGRKLRC; SEQ ID NO: 109. (The C-terminal cysteine residues are not part of the Gag sequence but are added to increase sensitivity in the ELISA.

For the ELISA, these 2 peptides were mixed together in PBS to a final concentration of 10 μg/ml each. This solution was used to coat ELISA plates. 50 μl was added to each well and left overnight (16 hours) at room temperature. The solution was then discarded and the plates were washed 3 times with PBS containing 0.05% Tween 20 (PBS/Twn). Plates were then blocked for 2 and a half hours with blocking buffer (5% low fat milk powder in PBS/Twn) at room temperature before washing again 3 times with PBS/Twn. Patient sera were added (50 μl, diluted 1 in 50 in blocking buffer) and the plate incubated for 2 hours at room temperature with occasional gentle mixing. The plates were then washed 3 times with PBS/Twn and incubated again with blocking buffer for 30 minutes before washing agin 3 times with PBS/Twn and addition of the secondary antibody, goat anti-human IgG conjugated to horse radish peroxidase (1:3000 dilution, Bio-Rad) for 1 hour at room temperature. The plates were then washed 7 times before developing.

We have also tested a CA peptide in an ELISA assay, WKVIPRKGERIRHSLTC; SEQ ID NO: 110.

### EXAMPLE 14

Detection of HRV5 in Patient Samples

Materials and Methods

Patients.

The study was approved by the Riverside Research Ethics Committee (RREC 0102) and all participating patients gave written consent. Synovial biopsy samples and peripheral blood samples were obtained from patients attending the Rheumatology clinic at Charing Cross Hospital, London, UK. Lymph node DNA samples were a kind gift from Dr Ruth Jarrett (LRF Glasgow). Blood DNA was examined from 37 individuals (17 with RA,3 OA, 17 normal).

DNA Extraction from Tissue Samples

Synovial biopsy tissue or labial salivary glands were processed immediately or stored at −20° C. Each sample was cut into 0.5-1 mm³ fragments with a sterile scalpel and incubated in 200μl of UV-irradiated lysis buffer (10 mM Tris-HCl (pH 8.3), 50 mM KCl, 2.5 mM MgCl₂, 0.5% v/v NP40, 0.5% v/v Tween) and Proteinase K added to a final concentration of 2 mg/ml for 48 hours at 56° C. The Proteinase K was then inactivated by heating to 95° C. for 15 minutes followed by centrifugation at 10,000× g for 10 minutes. The supernatant was stored in aliquots at −20° C. Suitability of the DNA for PCR was verified using primers for the single copy gene ERV-3 as described previously (Griffiths et al, J. Virol. 1997: 71: 2866-2872) DNA was extracted from peripheral blood lymphocytes using the DTAB/CTAB method (Gustincich et al Biotechniques. 1991; 11: 298-300) and analysed as described above.

Cloning a Sequence Tar for Sio-1

Approximately 10 mg of homogenized SS salivary gland lip biopsy were co-cultured with 10⁵ H9 cells in RPMI 1640 medium supplemented with 10% fetal calf serum (Biological Industries). The cultures were passaged twice weekly at a ratio of 1:8. After 14 days, the cells were homogenized using an ultra-Turrax T25 tissue grinder (IKA Labortechnik) at maximum speed and on ice. Cellular debris was removed by centrifugation at 4,000× g for 10 minutes at 4° C. The supernatant was then re-centrifuged at 20,000× g for 20 minutes at 4° C. to remove mitochondria and other sub-cellular organelles. The resultant supernatant was layered over a linear 20-65% (w/v) sucrose gradient. Sucrose gradients were prepared and run in a Beckman SW28 as described by Boyd et al., Lancet, (1989), ii:814-817. They were then centrifuged at 100,000 g for 16 hours. 1 ml fractions were collected and a 20 μl solution of RNA was prepared from these as follows: to 250 μl sucrose, 750 μl of RNAzol B (Biotecx Laboratories, Inc. Texas) is added followed by 125 μl chloroform. This mixture is then vortexed briefly, incubated on ice for 5 minutes and centrifuged at 13000× g, 4° C. for 15 minutes. Nucleic acids are then precipitated from the aqueous phase by addition of an equal volume of isopropanol and incubation on ice for 15 minutes. Precipitated RNA is pelleted by centrifugation (13000 g, 4° C. for 15 minutes) and the pellets washed in ice-cold 75% ethanol. Finally the RNA is resuspended in 20 μl water.

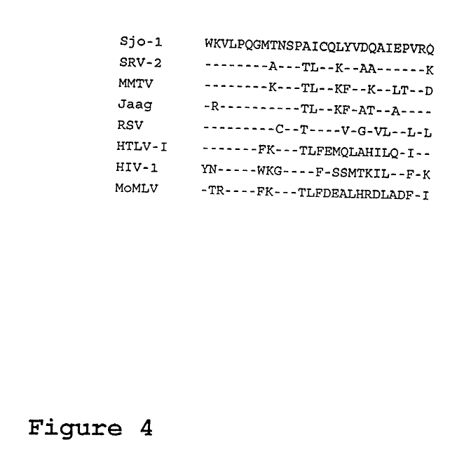

These solutions were subjected to reverse transcriptase-polymerase chain reaction (RT-PCR) using the degenerate pol primers described by Shih et al., J. Virol. (1989) 63: 64-75 which are capable of amplifying a wide variety of retroviral sequences. The products were then cloned into the pBluescript (KS-) plasmid (Stratagene) using standard methods (Sambrook et al 1989, Molecular Cloning 2nd ed. (Cold Spring Harbor Laboratory press)) and sequenced using an Applied Biosystems model 373A automated DNA sequence. A 126 bp clone was obtained and designated Sjo-1. Part of the deduced amino acid sequence of Sjo-1 is shown in FIG. 4 aligned with several other retroviral sequences (identified in a FASTA search of the entire Genbank and EMBL databases). The abbreviations and GenEMBL accession codes used in FIG. 4 are as follows.

SRV-2 Simian type D retrovirus serotype 2 (gb_vi:M16605)

MMTV Mouse mammary tumor virus (gb_vi:M15122)

Jaag Jaagsiekte sheep retrovirus (gb_vi:M80216)

RSV Rous sarcoma virus (gb_vi:Dl0652)

HTLV-1 Human T-cell leukemia virus type I (gb_vi:L10341)

HIV-1 Human immunodeficiency virus type 1 (gb_vi:D21166)

MOMLV Moloney murine leukemia virus (gb_vi:J02255)

The Sjo-1 sequence was detected in co-cultures after 14 days of co-cultivation and was obtained from fractions with a buoyant density of 1.15-1.16 g ml⁻¹ (the typical density of mature retroviral particles). This sequence was not detected in co-cultures which were passaged for longer than a month. It appears that no transfer of the virus occurred in these experiments. Moreover, it is believed that the co-cultivation, if indeed it plays a role, leads to stimulation of the cells which produce the virus.

The closest homologues of this short region as illustrated in FIG. 4 are the D and B-type retroviral sequences; SRV-2 (simian retrovirus serotype 2) and MMTV (mouse mammary tumor virus). Whilst the aligned region is too short for this comparison to be very meaningful, it did provide information which was useful in the design of subsequent primers for flanking regions such that these were biased towards B and D-type retroviral families. Sjo-1 could not be detected in H9 cells which had not been co-cultivated with SS salivary gland biopsy. Co-cultures from three individuals, two with primary SS and one with sicca syndrome were examined. Both the SS co-cultures were positive for Sjo-1 RNA whilst the sicca sample was negative.

Cloning a Larger Fragment of the Viral DNA

Degenerate primers for the active site of the protease (PR) gene of B and D-type retroviruses were designed. The PR gene, encoding as it does an enzyme, contains the second most highly conserved region of the retroviral genome. At the active site there is conservation of an aspartic acid-threonine-glycine (DTG) motif and this information was used in primer design. When these degenerate primers were used in conjunction with specific 3′ primers anchored within the Sjo-1 sequence, a further 806 bp of sequence was amplified from sucrose gradient purified viral RNA. The primers used were:

| | | |
|---|---|---|
| 1992 | (5′ GAGGTCATCCATGTAGTGTAAAATTTG 3′; | SEQ ID NO: 88) |
| 1784 | (5′ TAAAATTTGTACTTTTGGGCACTGCTG 3′; | SEQ ID NO: 89) |
| 3095 | (5′ TAGAYACKGGAGCWGATGT 3′; | SEQ ID NO: 90) |
| 3096 | (5′ IIIITAGAYACWGGRGCMGA 3′; | SEQ ID NO: 91) |

Where: K=G or T, M=A or C, R=A or G, W=A or T, Y=C or T and I=inosine.

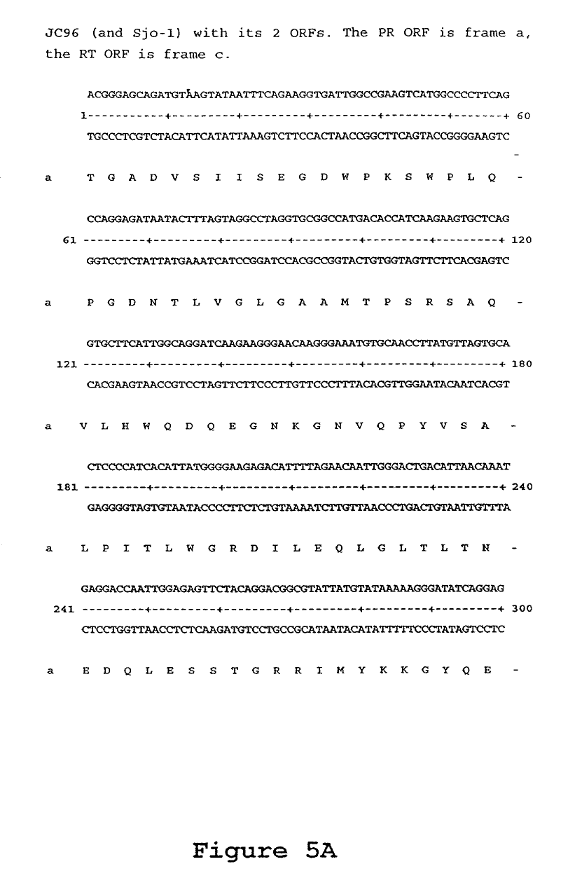

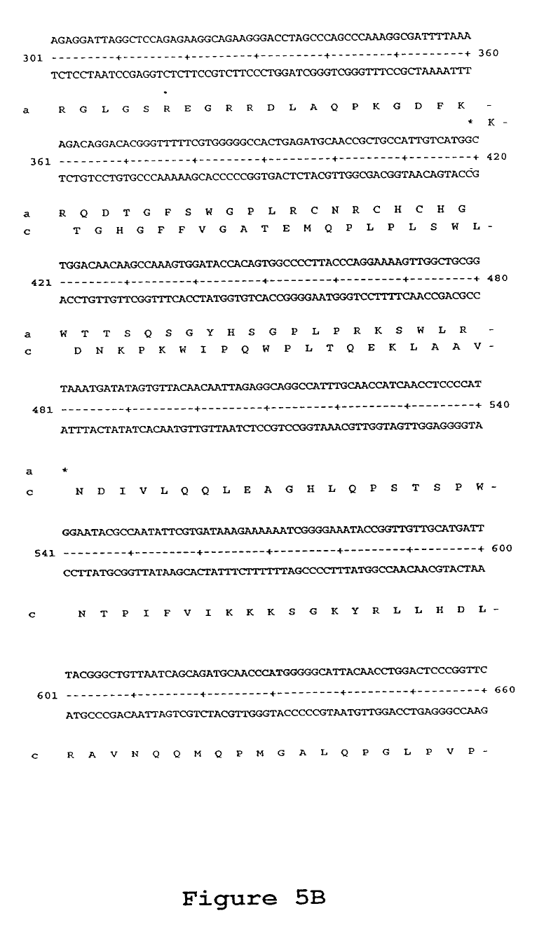

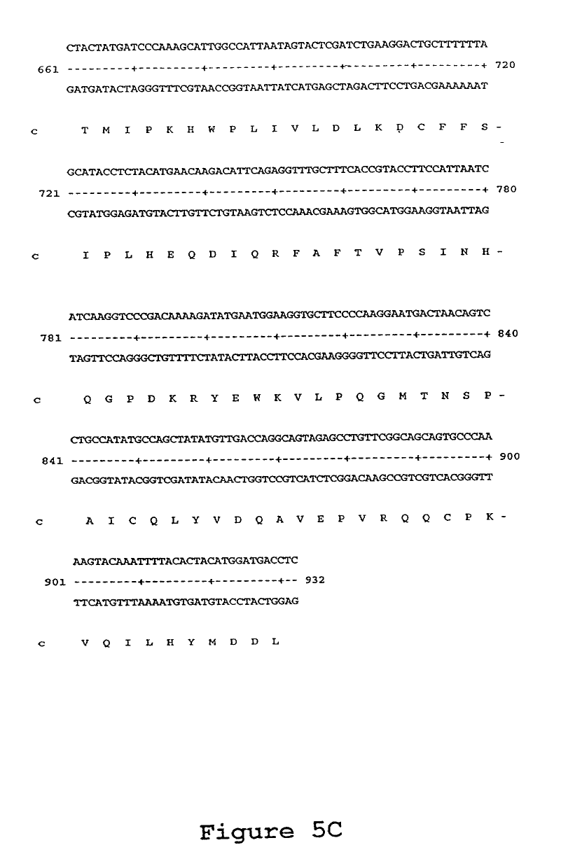

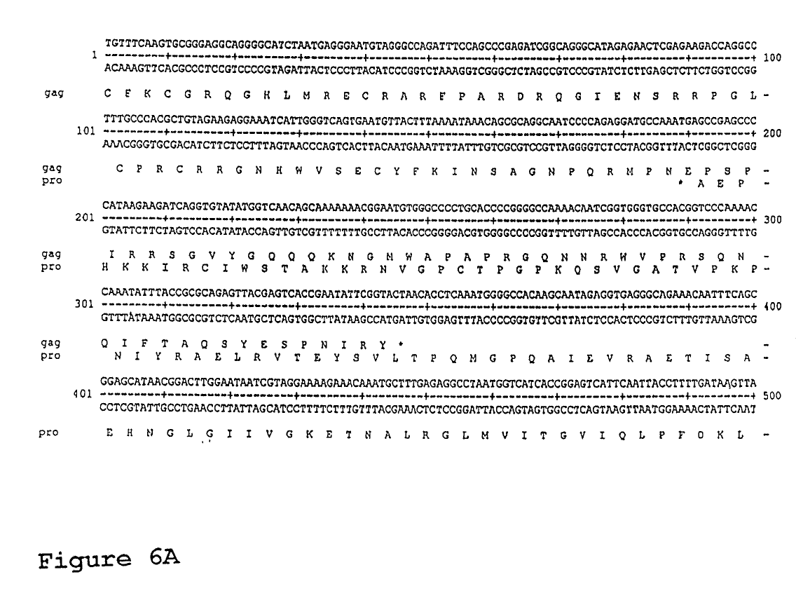

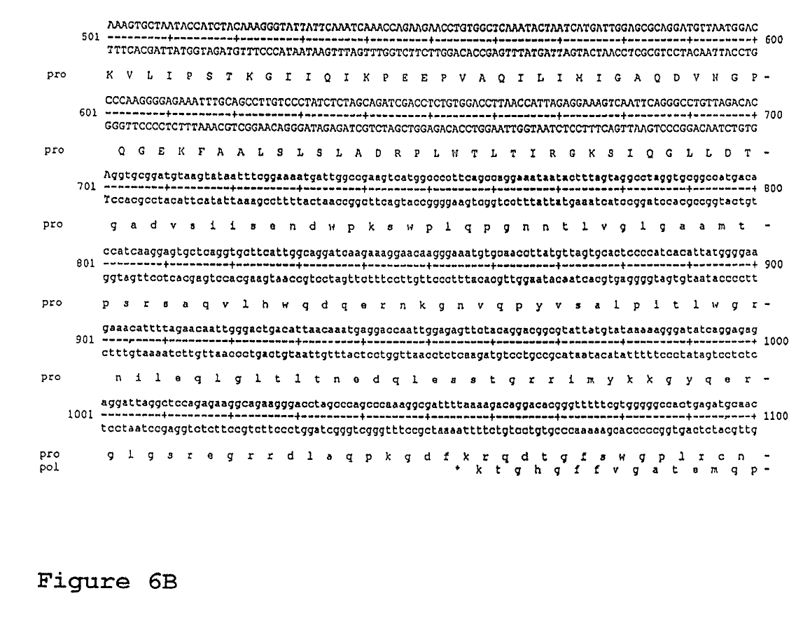

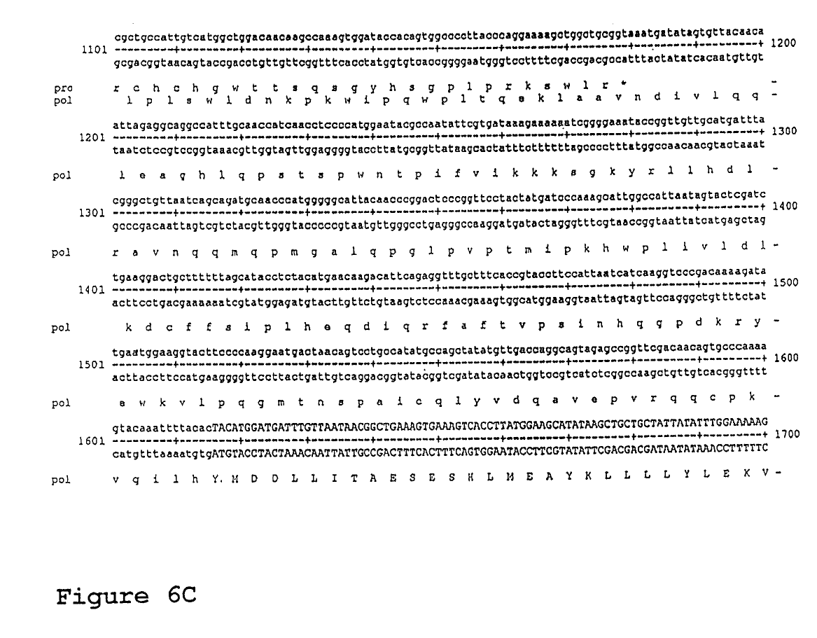

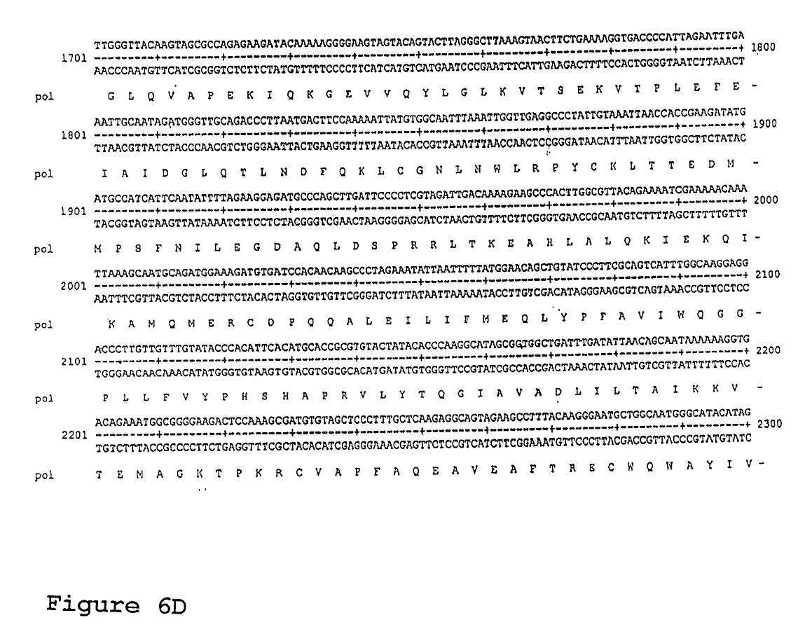

Synthesis of the cDNA was primed with 100 ng 1992, followed by PCR with 200 ng each of 1992 and 3096. Cycles were 94° C. 1 min, [94° C. 1 min; 50° C. 1 min; 72° C. 1 min 30 secs] for 25 cycles and a final extension at 72° C. for 7 mins. 1 μl of the 1st round product was transferred to a second PCR reaction using 200 ng each of primers 3095 and 1784. Cycles were [94° C. 1 min; 52° C. 1 min; 72° C. 1 min 30 secs] for 25 cycles followed by a final extension at 72° C. for 7 mins. The nucleotide sequence and the deduced amino acid sequence of this clone, designated JC96, are shown in FIG. 5. The two NcoI restriction sites are marked in bold. This fragment was removed to generate the positive control plasmid for PCR (pJC96ΔNco). Note that nucleotides 1-14 and 918-932 are derived from the degenerate primers used to clone Sjo-1 and JC96 and so may not represent the genuine sequence of this element in those regions.

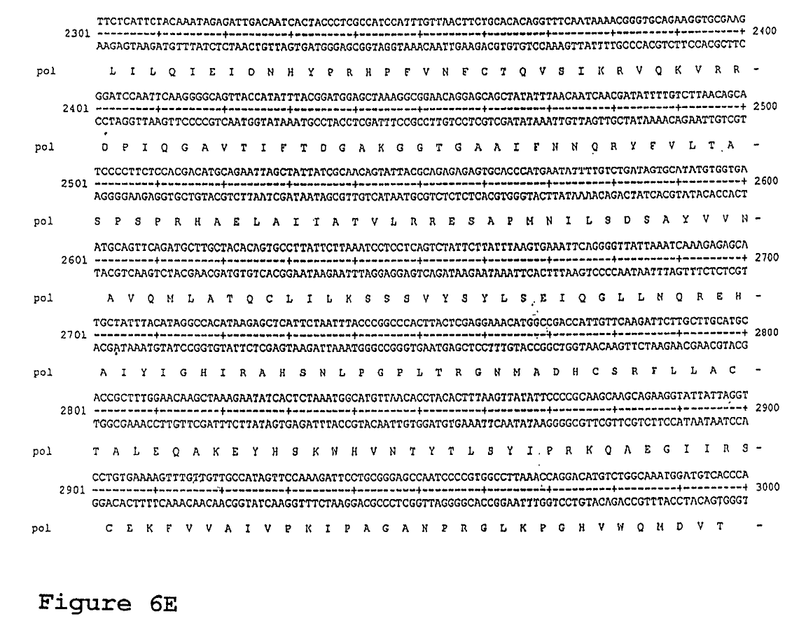

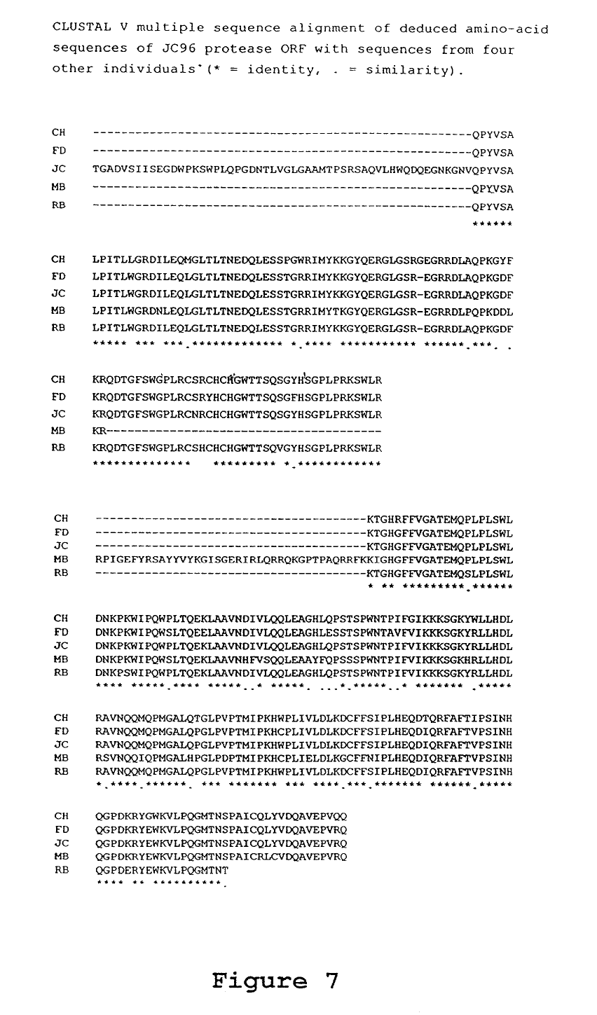

In FIG. 7, the following annotations have been used:

CH is from DNA of the submandibular gland of a patient with rheumatoid arthritis and secondary SS.

JC is the original clone from gradient fractionated RNA from a lip biopsy of a primary SS patient.

RB was cloned by RT-PCR from gradient fractionated RNA from the spleen of a primary SS patient with a B-cell lymphoma.

FD was cloned by RT-PCR from gradient fractionated RNA from the parotid gland of a non-SS subject.

MB was cloned by RT-PCR from gradient fractionated RNA from the submandibular gland of a non-SS subject.

The differences are mostly single base changes but there are also apparent insertions and deletions of bases that do not disrupt the ORF. One interesting observation is that the majority of differences between the five sequences occur in the CH sequence which unlike the other four sequences was amplified from DNA. A further observation from this data is that the PR ORF of one of the clones, MB, is truncated by 39 amino acids. This would render the virus non-viable if it were not compensated for by the RT ORF opening 39 amino acids earlier in this clone. The lengths of the overlap of the two ORFs are identical. The significance of these observations is at present unclear.

When considering these data attention should be drawn to the fact that PCR is notorious for giving false positive results due to cross-contamination of samples with the products of previous PCR experiments. This problem is greatly exacerbated when performing nested PCR experiments as here. Therefore in the abovementioned experiments, care was taken with controls to avoid false positives and to monitor the occurrence of cross-contamination. Specifically the measures taken were:

i) Separation of all experimental samples by including water controls which are taken through both stages of the nested reaction.

ii) UV cross-linking reaction mixes prior to the addition of template DNA.

iii) The use of a positive control which is a different size from the viral sequence. This was made by removing an internal NcoI fragment from the JC96 clone (indicated in FIG. 5).

Further Cloning of the Viral Genome.

From the above data provided by the present invention, it is clear that a “good” biopsy (i.e. one with a sufficiently high viral load) is required in order for HRV-5 to be cloned. Having established this, the present inventors have further provided sequence data and nested PCR primers which have been shown to detect proviral DNA in sample tissues. Therefore, even in the light of the fact that this element is present at very low levels in the specimens so far examined, and the fact that conventional methods (such as screening of a cDNA library prepared from infected tissue) cannot be used to clone the remainder of the virus the present invention provides materials and methods which would enable those skilled in the art to obtain further sequence data of HRV-5. Firstly inverse PCR methods as by Silver et al. (supra), Ochman et al, (supra) and Triglia et al, (supra) may be applied to genomic DNA obtained from an infected biopsy sample using primers disclosed herein or from the sequence data given in the figures. Secondly, viral sequences flanking the sequence data provided herein for HRV-5 may be amplified using degenerate PCR primers derived from other conserved regions of retroviral genomes in conjunction with primers specific for the HRV-5. Suitable primers are degenerate primers which work on a variety of retroviral sequences although they should be biased towards A, B and D-type sequences.

By coupling these primers with the specific primers described above, the cloned region of the genome could be expanded to include all but the long terminal repeat (LTR) and 3′ part of env. 5′ and 3′ rapid amplification of cDNA ends (RACE) (Frohman et al. 1988, Proc. Nat. Acad Sci. USA 85: 8998-9002) respectively can be used to clone these regions. The target material for these primers will be genomic DNA or RNA containing HRV-5 sequences, eg from inflamed synovia or blood from patients with SLE or RA. Indeed these the aforementioned regions have been cloned using the methods described in following examples.

### EXAMPLE 15

Cloning the 3′ Terminus of the HRV-5 Genome.

The final fragment of the HRV-5 genome was cloned using vectorette PCR on a Vectorette library constructed from Cla I digested DNA from bowel tissue from a patient with ulcerative colitis.

First stage primers:

HRV-5 pol primer (IN2F1)

5′-CACGTCACTGTAGATACATATTCAG-3′; SEQ ID NO: 111

Vectorette primer (Genosys)

Conditions were 40 cycles of 94° C., 1 minute 10 seconds; 60° C., 1 minute 10 seconds; 72° C., 3 minutes with initial denaturation at 94° C. for 4 minutes. 1 μl of the first stage products were transferred to the second round reaction.

Primers:

HRV-5 pol primer (IN2F2)

5′-GGTGTAGTTATGGCCACAGCCATG; SEQ ID NO: 112.

Vectorette nested primer (Genosys)

Conditions for this PCR were as for the first stage. In this experiment a third round of PCR was required to obtain a clean product and 1 pl of the second stage products was transferred to a third stage reaction containing primers IN2F3 and a vectorette primer.

Primers

HRV-5 pol primer (IN2F3)

5′-AACACTGCTTGCAGGCTTTTGCAG-3′ SEQ ID NO: 113.

Vectorette sequencing primer (used as a third stage primer)

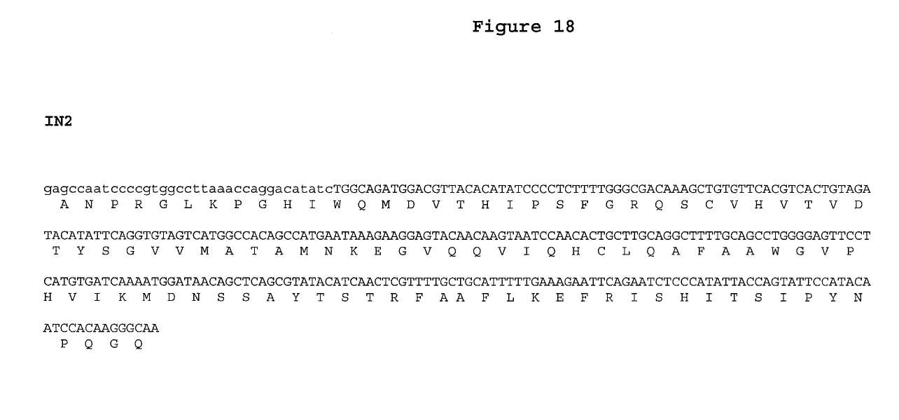

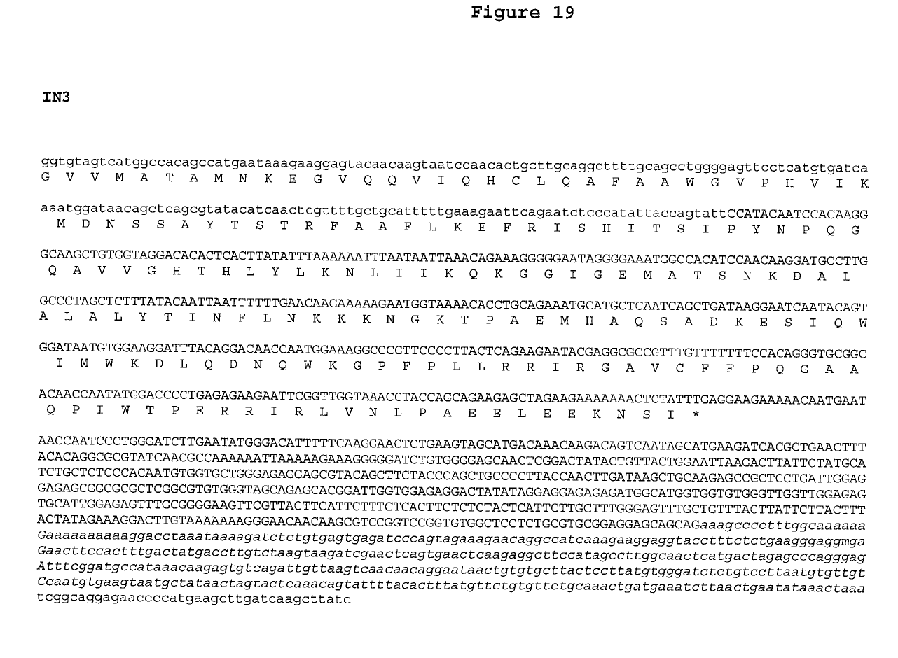

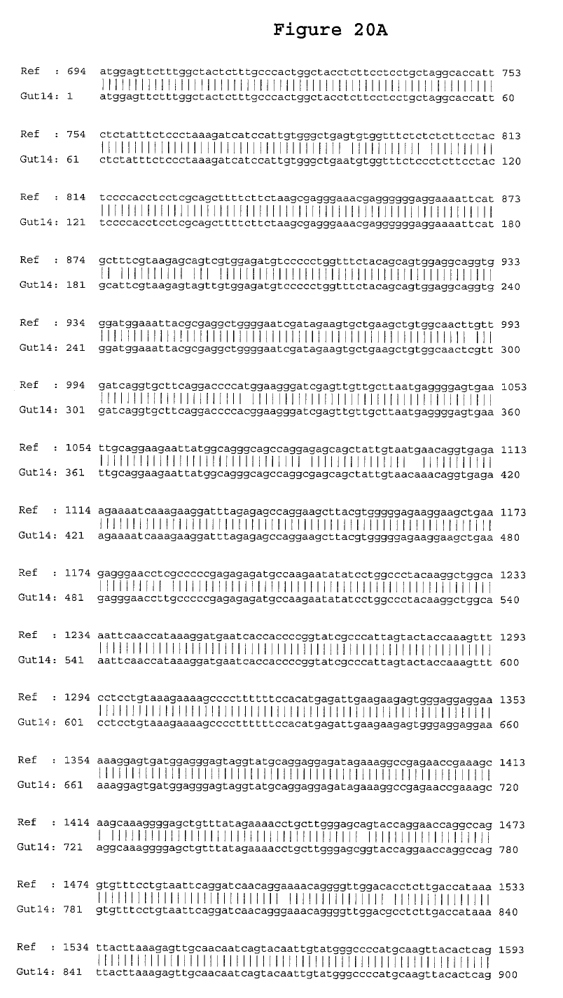

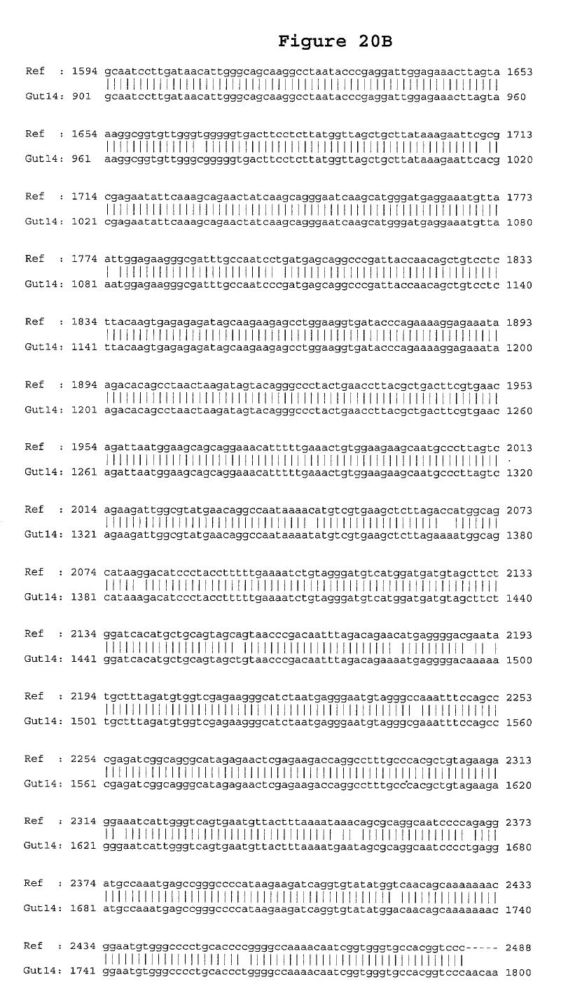

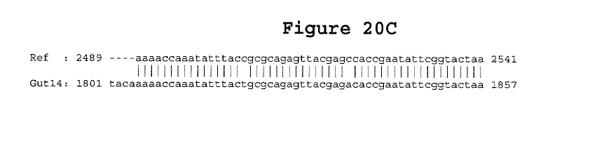

Conditions for this stage were 35 cycles of 94° C., 1 minute 10 seconds; 52° C., 1 minute 10 seconds; 72° C., 2 minutes with an initial denaturation at 94° C. for 4 minutes. Analysis of the products by agarose gel electrophoresis revealed a single product of 1455 bp. Sequencing revealed this to contain 997 bp of HRV-5 sequence comprising the remainder of the pol gene and the 3′ LTR. The fragment also included 458 bp of flanking genomic (i.e., non-HRV-5) DNA. Thus in this reaction the vectorette PCR allowed the amplification of the 3′ virus integration site. See FIGS. 18 and 19.

### EXAMPLE 16

Amplification of the 5′ Long Terminal Repeat

Confirmation that the 3′ terminal region of HRV-5 was also present 5′ to gag was provided by PCR with specific primers in a hemi-nested PCR.

Primers:

| | | |
|---|---|---|
| U3F1 | 5′-CTGTGGGGAGCAACTCGGACTATAC; | SEQ ID NO: 114 |
| G2R1 | 5′-GCTTCCTGGCTCTCTAAATCCTTC; | SEQ ID NO: 115 |
| G2R2 | 5′-CTCACCGGTTCATTACAATAGCTGC; | SEQ ID NO: 116. |

PCR was performed on DNA extracted from a blood sample from a normal individual and colon tissue from a patient with ulcerative colitis. PCRs were performed using the Expand High Fidelity PCR System for Roche Molecular as recommended. First stage PCR was with primers U3F1 and G2R1. The conditions were 40 cycles of 94° C., 1 minute 20 seconds; 52° C., 1 minute 20 seconds; 68° C., 2 minutes with an initial denaturation at 94° C. for 4 minutes. One microlitre of the first round products were transferred to a second round hemi-nested PCR with primers U3F1 and G2R2. Conditions were as for the first round. A 1100 bp product was amplified from both samples. Sequencing confirmed that the expected LTR region (initially cloned as part of IN3) was present upstream of the gag sequence. In addition, the products of this PCR included fragment Gag7, Gag6, Gag5, Gag4 and part of Gag2, confirming that these regions are contiguous in the HRV-5 genome.

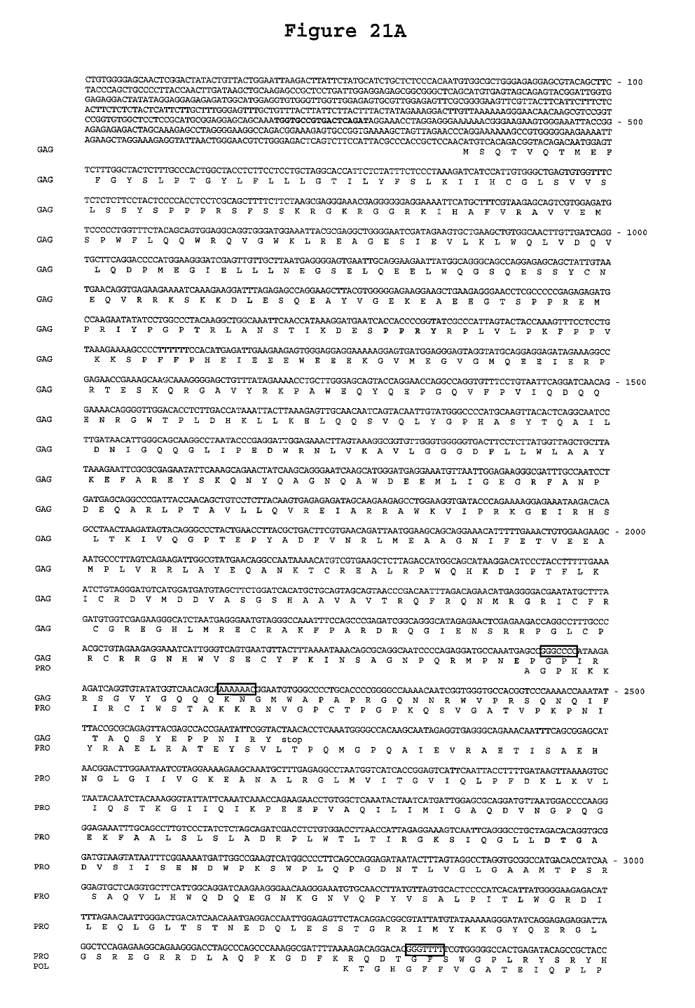

A full length HRV-5 clone was then constructed from the various PCR amplified fragments with the structure LTR-gag-pro-pol-LTR. See FIG. 21.

The cloning of the HRV5 LTR will facilitate methods for assessing expression and tropism of HRV5 in different cell types.

Reporter plasmids were constructed by amplifying the LTR of HRV-5 using the PCR and cloning into the luciferase reporter vector pGL-3 (Promega). LTR fragments were amplified in 50 μl reaction volumes using the Expand high fidelity PCR system (Roche Molecular) as recommended with primers LTR-F (5′-CTGTGGGGAG CAACTCGGACTATAC; SEQ ID NO: 114) and LTR-R (5′-CTTGCTGCTCCTCCGCACGCGG; SEQ ID NO: 117) using the plasmid pHRV56 as a template. PCR fragments were gel purified and blunt-end cloned into Sma I digested pGL-3 using standard procedures. Candidate clones were sequenced to confirm the identity and orientation of the LTR insert.

The ability of the HRV-5 LTR to drive expression of the luciferase reporter gene was then tested using the Dual-Luciferase Reporter Assay System (Promega) as recommended. This assay system measures the HRV-5 LTR activity relative to the activity of the SV40 early promoter (which is active in most cell types) in the same cell type. For example, in the 293-T cell line (a human embryonic kidney epithelial line) HRV-5 LTR had 39% of the activity of the SV40 early promoter under the assay conditions used.

### EXAMPLE 17

A. Expression of HRV-5 Gag in Mammalian Cells

A diagnostic assay for HRV-5 infection can be based on indirect immunofluorescence assays (IFA) to detect anti-HRV5 antibodies present in patient serum samples. As we have not yet established a culture system for HRV-5, we have expressed the HRV-5 Gag polyprotein as a recombinant antigen in the human embryonic kidney epithelial cell line 293-T.

The HRV-5 gag gene was PCR amplified from human genomic DNA as follows:

Primers were:

| | | |
|---|---|---|
| F1 | 5′-TAGGAAAGAGGTATTTACTGG; | SEQ ID NO: 118 |
| R1 | 5′-ATCACGAATATTGGCGTATTCCATGG; | SEQ ID NO: 119 |
| F2 | 5′-GGGAGACTGTCTTCCACTACG; | SEQ ID NO: 120 |
| R2 | 5′-TGATGGTTGCAAATGGCCTGCCTC; | SEQ ID NO: 121. |

First round PCR reaction mixtures contained 10 pmol of F1 and R1 primer, 25 mM of each dNTP (Pharmacia), 2.5 mM MgCl₂, 2.5 U of Taq polymerase in PCR buffer number 3 (Expand Long Template System, Roche Molecular), and 100 ng of DNA from bowel tissue of a patient with ulcerative colitis in a final volume of 50 μl. PCR cycling conditions were as follows: 3 min at 94° C.; 30 cycles, each consisting of 1 min at 94° C., 1 min at 51°, and 3min at 72° C. 1 μl of the first round products were transferred to a second stage PCR containing 10 pmol of F2 and R2 primer, 25 mM of each dNTP (Pharmacia), 2.5 mM MgCl₂, 2.5 U of Taq polymerase in PCR buffer number 3 (Expand Long Template System, Roche Molecular), in a final volume of 50 μl. PCR cycling conditions were as for the first round of PCR. All the PCR amplifications were performed on a MJ research PTC200 apparatus, Peltier Thermal cycler. Analysis of second round PCR products on 1% TAE agarose gel revealed 3 kb and 0.9 kb bands.

Gag-PR PCR products were separated on an agarose gel, and the 3 kb PCR products were purified using the QIAquick Gel Extraction kit (Qiagen). Purified PCR products were blunt-end cloned into EcoR V-digested pBlueScript KS+ vector. This plasmid is named pBlue-gag124.

The HRV-5 Gag gene was then subcloned from pBlue-gag124 into pcDNA3.1+ (Invitrogen) using Nhe I and BamH I restriction sites contained in the PCR primers to generate plasmid pcDNA3.1+/HRV-5 Gag. Then, the GFP coding region was inserted into this plasmid as a C-terminal tag downstream of gag, using BamH I and Xba I restriction sites to create plasmid pcDNA3.1+/HRV-5 Gag-GFP.

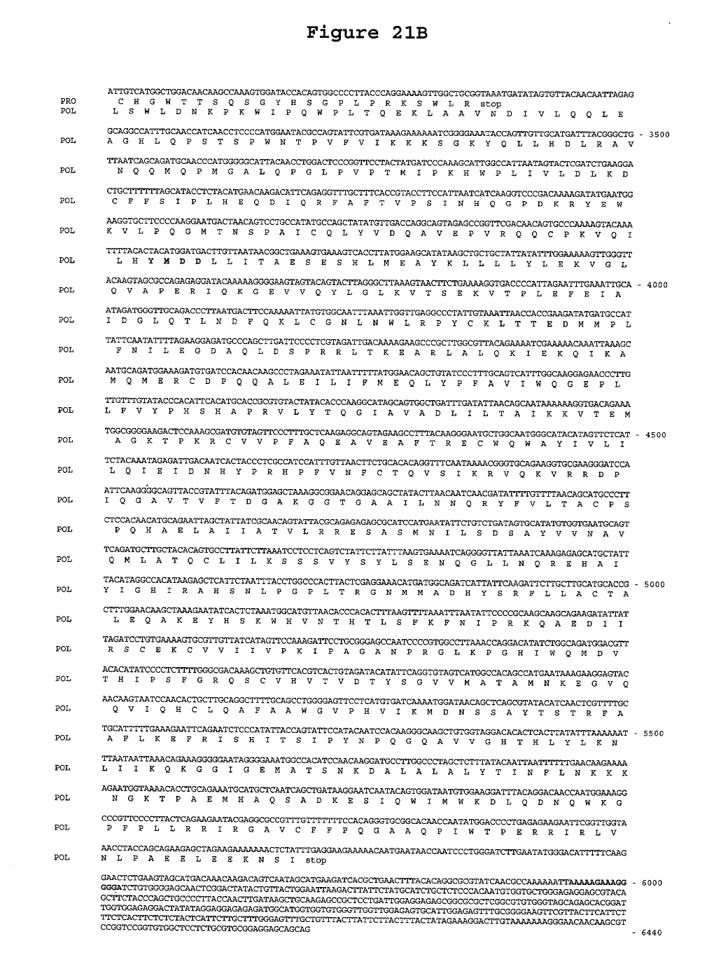

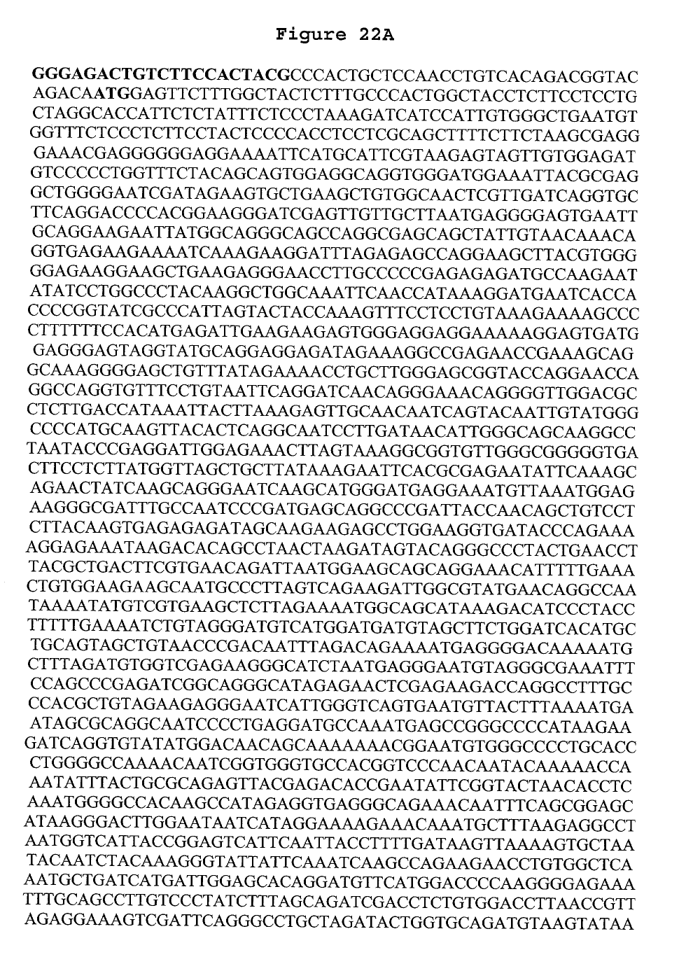

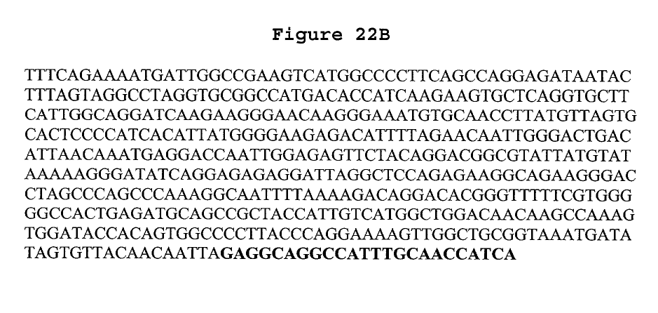

Cloned PCR products were sequenced using an Applied Biosystems 373A automated DNA sequencer. Computer-aided analysis of protein and nucleotide sequences was performed with Sequencher program (FIG. 22).

Transfections were performed in six-well plates (Greiner). Cells were passaged the day before preparation of the six-well plates. On the day of the transfection, the cells were ˜70% confluent. Transfections were carried out with Lipofectamine (Gibco BRL) in accordance with the user's manual. The total amount of plasmid DNA used in transfections was 1.6 ug for each well, in a final volume of 1 ml of OPTIMEM 1 (Gibco BRL, ref 31985-047) for 5 to 6 hours at 37° C., after which the transfected cells were washed twice with DMEM, and the medium was changed to DMEM containing fetal calf serum. The cells were processed for further studies 24 to 48 hours after transfection.

HRV-5 Gag proteins can be expressed using any standard mammalian expression vector (e.g, pcDNA3.1, Invitrogen) using methods well known in the art.

However, in preliminary experiments using pcDNA3.1+/HRV-5Gag-GFP, only a very low level of Gag-GFP protein expression was observed. To obtain a higher expression level, we used an inducible mammalian expression system to express HRV-5 Gag-GFP fusion proteins. This system (Geneswitch) contains an intron between the promoter and the HRV-5 Gag coding sequence.

The “Geneswitch” System (Invitrogen) is an inducible expression system with a transcription control mechanism that offers minimal levels of basal expression in mammalian cells. The “Geneswitch” expression vector pGene/V5-His provides a hybrid promoter sequence, GAL4 UAS/E1b consisting of a 10-base pair TATA box sequence from the Adenovirus E1b gene and six binding sites for the yeast GAL4 protein. Without additional factors, the GAL4 UAS/E1b sequence is transcriptionally silent, yielding the lowest possible level of basal expression.

For inducible expression, the addition of mifepristone activates transcription from the GAL4 UAS/Elb promoter and expression of the desired protein, in this case the HRV-5 Gag-GFP fusion protein.

Plasmid constructs

Primers used:

F7-SEQ ID NO: 122
ATAAGAATGCGGCCGCTAAACTATGCCATGGAGTTCTTTGGCTACTCTTTG
R7-SEQ ID NO: 123
ATAGTTTAGCGGCCGCATTCTTATGGTACCGAATATTCGGTGTCTCGTAAC
F9-SEQ ID NO: 124
ATAAGAATGCGGCCGCTAAACTATGCCATGGTGAGCAAGGGCGAGGAGCTG
TTCACCTTCACC
R9- SEQ ID NO: 125
ATAGTTTAGCGGCCGCATTCTTATGCTTGTACAGCTCGTCCATGCCGAG
R1O- SEQ ID NO: 126
ATAGTTTAGCGGCCGCATTCTTATTTACTTGTACAGCTCGTCCATGCCGAG

The HRV-5 Gag gene was amplified from gagl24-pBlue plasmid, using F7 and R7 primers. The GFP gene was amplified from pCDNA3.1-emerald plasmid using F9 and R9 primers. The HRV-5 Gag-GFP fusion fragment was amplified from pcDNA3.1+/HRV-5 Gag-GFP using F7 and R10 primers. PCR reactions were performed as described above.

GFP, HRV-5 Gag and HRV-5 Gag-GFP PCR products were cloned into pGene/V5-His using a Not I restriction site engineered into the primer. HRV-5 Gag and GFP genes were cloned in frame with V5-His Tag in their C-terminus. The constructs are called pGene/HRV-5-Gag-GFP, pGene/GFP, pGene/HRV-5 Gag.

Transfections were performed in 293T cells plated in six-well plates (Greiner) as described above. 293-T cells were co- transfected with pSwitch and pGene/HRV-5 Gag-GFP, pGene/GFP. The day after transfection, mifepristone was added to the culture medium to a final concentration of 20 nm. The total amount of DNA used in transfections was 1.6 μg of pGene constructs and 0.4 μg of pSwitch plasmid. The cells were processed for further studies 24 hours after transfection.

Eight hours after induction, green cells were already visible. Twenty-four hours after transfection, about 20% of transfected cells are green indicating expression of HRV-5-Gag-GFP proteins.

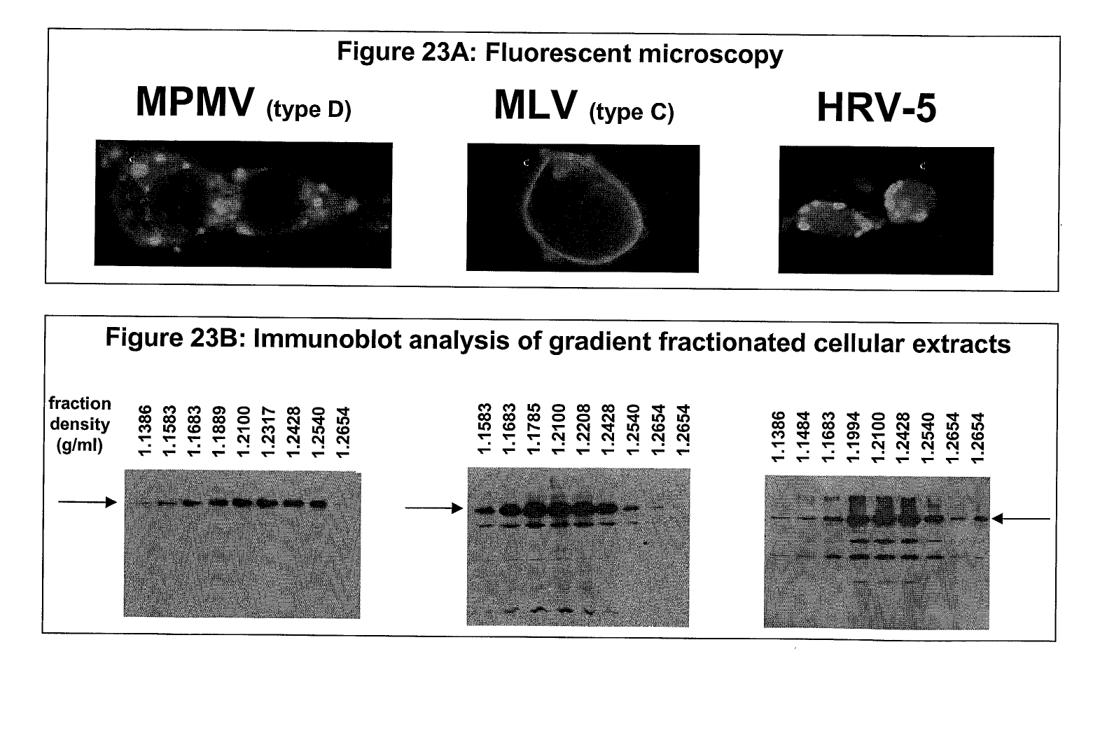

HRV-5 Gag-GFP proteins are localized in the cytoplasm of transfected cells with a fairly homogeneous distribution (FIG. 23A). Immunoblot analysis of cell lysates prepared from 293T cells transfected with pGene/HRV-5 Gag-GFP showed a strong 25 kDa protein using monoclonal anti-GFP antibodies (JL8) (FIG. 23B).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A retrovirus which comprises a nucleotide sequence as shown in FIG. 21A (SEQ ID NO: 127) and variants, mutants and fragments thereof.

2. A retroviral vector comprising the retrovirus of claim 1, further comprising a cloning site for insertion of a heterologous nucleic acid molecule.

3. The retroviral vector of claim 2, wherein said heterologous nucleic acid molecule encodes a protein selected from the group consisting of cytokines, herpes simplex virus type 1 thymidine kinase, adenosine deaminase, iduronate-2-sulfatase, low density lipoprotein receptor, and cystic fibrosis transmembrane conductance regulator.

4. A method for expressing a heterologous nucleic acid sequence in a mammalian cell, comprising introducing, into chromosomal DNA of mammalian cells isolated in culture, a first retroviral vector comprising:
(i) HRV-5 LTR sequences for integration of the vector into chromosomal DNA of the mammalian cell;
(ii) a heterologous nucleic acid sequence to be expressed in the mammalian cell operably linked to said LTR; and
(iii) a second retroviral vector which functions as a helper virus to facilitate packaging and replication of said first retroviral vector, thereby expressing said heterologous nucleic acid in said mammalian cell.

5. The method of claim 4, further comprising a step of detecting a change in phenotype of the mammalian cell as a result of expression of the heterologous nucleic acid.

6. The method of claim 4, wherein said heterologous nucleic acid encodes a protein selected from the group consisting of cytokines, herpes simplex virus type 1 thymidine kinase, adenosine deaminase, iduronate-2-sulfatase, low density lipoprotein receptor, and cystic fibrosis transmembrane conductance regulator.

7. A method for identifying therapeutic agents useful for the treatment of HRV-5 infection, said method comprising:
(i) providing two host cell populations comprising a reporter construct containing the HRV-5 LTR operably linked to a reporter gene;
(ii) contacting a first host cell population with an agent suspected of modulating HRV-5 LTR activity; and
(iii) comparing expression levels of said reporter gene in the presence and absence of said agent, thereby identifying agents which alter HRV5 LTR activity.

8. The method of claim 7, wherein said reporter gene is selected from the group consisting of luciferase, green fluorescent protein, and chloramphenicol acetyl transferase.

9. A peptide for use in the production of anti-HRV5 immunospecific antibodies selected from the group consisting of SFSSKRGKRGGRKIHC (SEQ ID NO: 108); PWFLQQWRQVGRKLRC (SEQ ID NO: 109); and WKVIPRKGERIRHSLTC (SEQ ID NO: 110).

10. An HRV-5 polypeptide as shown in FIG. 21A (SEQ ID NO: 103).

11. A nucleic acid molecule encoding the polypeptide of claim 10.

12. An HRV-5 Gag3 polypeptide as shown in FIG. 14 (SEQ ID NO: 93).

13. A nucleic acid molecule encoding the polypeptide of claim 12.

14. An HRV-5 Gag4 polypeptide as shown in FIG. 15 (SEQ ID NO: 95).

15. A nucleic acid molecule encoding the polypeptide of claim 14.

16. An HRV-5 Gag5 polypeptide as shown in FIG. 16 (SEQ ID NO: 97).

17. A nucleic acid molecule encoding the polypeptide of claim 16.

18. An HRV-5 IN2 polypeptide as shown in FIG. 18 (SEQ ID NO: 100).

19. A nucleic acid molecule encoding the polypeptide of claim 18.

20. An HRV-5 IN3 polypeptide as shown in FIG. 19 (SEQ ID NO: 102).

21. A nucleic acid molecule selected from the group consisting of:
1)(N(0-200) 5′-TTGGAGCGGTGGGCGTARTGGAAGG-N(0-200)3′;
2) (N(0-200) 5′-CACGTCACTGTAGATACATATTCAG-N(0-200)3′;
3) (N(0-200) 5′-GGTGTAGTTATGGCCACAGCCATG-N(0-200)3′;
4) (N(0-200) 5′-AACACTGCTTGCAGGCTTTTGCAG-N(0-200)3′;
5) (N(0-200) 5′-CTGTGGGGAGCAACTCGGACTATAC-N(0-200)3′;
6) (N(0-200) 5′-GCTTCCTGGCTCTCTAAATCCTTC-N(0-200)3′;
7) (N(0-200) 5′-CTCACCGGTTCATTACAATAGCTGC-N(0-200)3′;
8)(N(0-200) 5′-TAGGAAAGAGGTATTTACTGG-N(0-200)3′;
9)(N(0-200) 5′-ATCACGAATATTGGCGTATTCCATGG-N(0-200)3′;
10)(N(0-200) 5′-GGGAGACTGTCTTCCACTACG-N(0-200)3′;
11)(N(0-200) 5′-TGATGGTTGCAAATGGCCTGCCTC-N(0-200)3′;
12)(N(0-200) 5′ATAAGAATGCGGCCGCTAAACTATGCCATGGAGTTCTTT GGCTACTCTTTG-N(0-200)3′;
13) (N(0-200) 5′ATAGTTTAGCGGCCGCATTCTTATGGTACCGAATATT CGGTGTCTCGTAAC-N(0-200)3′;
14) (N(0-200) 5′ATAAGAATGCGGCCGCTAAACTATGCCATGGTG AGCAAGGGCGAGGAGCTGTTCACCTTCACC
15) (N(0-200) 5′ATAGTTTAGCGGCCGCATTCTTATGCTTGTACAGCTC GTCCATGCCGAG-N(0-200)3′;
16) (N(0-200) 5′ATAGTTTAGCGGCCGCATTCTTATTTACTTGTACAGCTCGTC CATGCCGAG-N(0-200)3′.
